SEQUENCE LISTING (14 sequences)

SEQ ID NO: 1 - protein, 669 aa, Staphylococcus aureus
MMKKIKIVPLILIVVVVGFGIYFYASKDKEINNTIDAIEDKNFKQVYKDSSYISKSDNGEVEMTERPIKIYNSLGVKDINIQDRKIKKVSKNKKRVDAQYKIKTNYGNIDRNVQFNFVKEDGMWKLDWDHSVIIPGMQKDQSIHIENLKSERGKILDRNNVELANTGTAYEIGIVPKNVSKKDYKAIAKELSISEDYIKQQMDQNWVQDDTFVPLKTVKKMDEYLSDFAKKFHLTTNETESRNYPLGKATSHLLGYVGPINSEELKQKEYKGYKDDAVIGKKGLEKLYDKKLQHEDGYRVTIVDDNSNTIAHTLIEKKKKDGKDIQLTIDAKVQKSIYNNMKNDYGSGTAIHPQTGELLALVSTPSYDVYPFMYGMSNEEYNKLTEDKKEPLLNKFQITTSPGSTQKILTAMIGLNNKTLDDKTSYKIDGKGWQKDKSWGGYNVTRYEVVNGNIDLKQAIESSDNIFFARVALELGSKKFEKGMKKLGVGEDIPSDYPFYNAQISNKNLDNEILLADSGYGQGEILINPVQILSIYSALENNGNINAPHLLKDTKNKVWKKNIISKENINLLTDGMQQVVNKTHKEDIYRSYANLIGKSGTAELKMKQGETGRQIGWFISYDKDNPNMMMAINVKDVQDKGMASYNAKISGKVYDELYENGNKKYDIDE

SEQ ID NO: 2 - protein, 665 aa, Staphylococcus aureus
MKKIYISVLVLLLIMIIITWLFKDDDIEKTISSIEKGNYNEVYKNSSEKSKLAYGEEEIVDRNKKIYKDLSVNNLKITNHEIKKTGKDKKQVDVKYNIYTKYGTIRRNTQLNFIYEDKHWKLDWRPDVIVPGLKNGQKINIETLKSERGKIKDRNGIELAKTGNTYEIGIVPNKTPKEKYDDIARDLQIDTKAITNKVNQKWVQPDSFVPIKKINKQDEYIDKLIKSYNLQINTIKSRVYPLNEATVHLLGYVGPINSDELKSKQFRNYSKNTVIGKKGLERLYDKQLQNTDGFKVSIANTYDNKPLDTLLEKKAENGKDLHLTIDARVQESIYKHMKNDDGSGTALQPKTGEILALVSTPSYDVYPFMNGLSNNDYRKLTNNKKEPLLNKFQITTSPGSTQKILTSIIALKENKLDKNTNFDIYGKGWQKDASWGNYNITRFKVVDGNIDLKQAIESSDNIFFARIALALGAKKFEQGMQDLGIGENIPSDYPFYKAQISNSNLKNEILLADSGYGQGEILVNPIQILSIYSALENNGNIQNPHVLRKTKSQIWKKDIIPKKDIDILTNGMERVVNKTHRDDIYKNYARIIGKSGTAELKMNQGETGRQIGWFVSYNKNNPNMLMAINVKDVQNKGMASYNATISGKVYDDLYDNGKTQFDIDQ

SEQ ID NO: 3 - protein, 8 aa, Staphylococcus aureus
IYNSLGVK

SEQ ID NO: 4 - protein, 7 aa, Staphylococcus aureus
DINIQDR

SEQ ID NO: 5 - protein, 10 aa, Staphylococcus aureus
ELSISEDYIK

SEQ ID NO: 6 - protein, 12 aa, Staphylococcus aureus
FQITTSPGSTQK

SEQ ID NO: 7 - protein, 11 aa, Staphylococcus aureus
ILTAMIGLNNK

SEQ ID NO: 8 - protein, 11 aa, Staphylococcus aureus
YEVVNGNIDLK

SEQ ID NO: 9 - protein, 8 aa, Staphylococcus aureus
VALELGSK

SEQ ID NO: 10 - protein, 8 aa, Staphylococcus aureus
SYANLIGK

SEQ ID NO: 11 - protein, 11 aa, Staphylococcus aureus
LAYGEEEIVDR

SEQ ID NO: 12 - protein, 10 aa, Staphylococcus aureus
SYNLQINTIK

SEQ ID NO: 13 - protein, 9 aa, Staphylococcus aureus
ILTSIIALK

SEQ ID NO: 14 - protein, 8 aa, Staphylococcus aureus
IALALGAK

(19) United States
(12) Patent Application Publication
(10) Pub. No.: US 2025/0075247 A1
(43) Pub. Date: Mar. 6, 2025

(54) METHOD FOR DETERMINING THE METHICILLIN RESISTANCE OF STAPHYLOCOCCUS AUREUS STRAINS

(71) Applicants: UNIVERSITE CLAUDE BERNARD LYON 1, VILLEURBANNE (FR); ECOLE NORMALE SUPERIEURE DE LYON, LYON (FR); HOSPICES CIVILS DE LYON, LYON (FR); CENTRE NATIONAL DE LA RECHERCHE SCIENTIFIQUE (CNRS), Paris (FR); INSERM (institut National de la Santé et de la Recherche Médicale), Paris (FR); WEEZION, VERGISSON (FR)

(72) Inventors: Jérôme LEMOINE, Lucenay (FR); Francis DEFORET, LYON (FR); François VANDENESCH, RILLIEUX-LA-PAPE (FR); Olivier DAUWALDER, LYON (FR)

(73) Assignees: UNIVERSITE CLAUDE BERNARD LYON 1, VILLEURBANNE (FR); ECOLE NORMALE SUPERIEURE DE LYON, LYON (FR); HOSPICES CIVILS DE LYON, LYON (FR); CENTRE NATIONAL DE LA RECHERCHE SCIENTIFIQUE (CNRS), PARIS (FR); INSERM (Institut National de la Santé et de la Recherche Médicale), PARIS (FR); WEEZION, VERGISSON (FR)

(21) Appl. No.: 18/728,513

(22) PCT Filed: Jan. 11, 2023
(86) PCT No.: PCT/FR2023/050038

§ 371 Date: Jul. 12, 2024

(30) Foreign Application Priority Data
Jan. 13, 2022 (FR) FR2200268

(51) Int. Cl. C12Q 1/18; C12Q 1/37; G01N 33/68

(52) U.S. Cl. CPC C12Q 1/18; C12Q 1/37; G01N 33/6848; G01N 2333/31; G01N 2333/976

(57) ABSTRACT

The present invention relates to a method for determining the methicillin-resistance properties of a Staphylococcus aureus strain present in a biological sample, the method comprising the following steps: •a) incubating the biological sample containing the Staphylococcus aureus strain for at least 15 minutes in the presence of an antibiotic from the class of beta-lactams selected from the following group: cefoxitin and 6-APA (6-aminopenicillanic acid); •b) isolating the bacteria present in the biological sample; •c) lysing the bacteria and hydrolysing the bacterial proteins in order to obtain a mixture of peptides; and •d) analysing this mixture of peptides by targeted mass spectrometry, the detection of at least one peptide originating from the protein PBP2a (SEQ ID NO. 1) or PBP2c (SEQ ID NO. 2) during this analysis step being indicative of the methicillin resistance of the Staphylococcus aureus strain present in the biological sample.

### FIELD OF THE INVENTION

The present invention concerns a method for characterizing bacterial strains belonging to the staphylococcus family, and, in particular, to a method for identifying bacterial strains resistant to certain antibiotics. This allows healthcare personnel to select appropriate antibiotics for each infected patient, based on this resistance data.

### STATE OF THE ART

The increased use of antibiotics in recent decades has led to the emergence of resistance mechanisms in numerous bacterial species.

The first penicillin-resistant strains of Staphylococcus aureus were characterized only two years after the introduction of this antibiotic into the treatment chain for bacterial infections (Kirby, 1944) and spread rapidly, leading to the gradual discontinuation of the use of penicillin in clinical practice in favour of new semi-synthetic antibacterial molecules such as methicillin and oxacillin.

Then in the 1960s, new strains of Staphylococcus aureus resistant to methicillin, as well as to other antibiotic compounds, appeared. These strains are designated by the acronym MRSA for methicillin resistant Staphylococcus aureus.

These MRSA strains express a particular toxin, Panton-Valentine leukocidin (PVL). This so-called pore-forming toxin induces pores in the cell walls, especially cells of the immune system, thus killing the cells capable of fighting infection.

Although initially confined to the hospital environment, MRSA strains then spread into the population. In 2019, in Europe, it was estimated that MRSA strains represent between 1% and 46% of Staphylococcus aureus infections. According to the World Health Organization, a person infected by a MRSA strain has a 64% higher probability of death than a person infected with a non-resistant strain.

The detection of MRSA strains is therefore a major challenge for the management and treatment of patients with Staphylococcus aureus infections.

The MRSA phenotype is due to the expression of a specific protein, penicillin binding protein 2a (PBP2a), encoded by the MecA gene, and, in rarer cases, to the expression of the PBP2c protein encoded by the MecC gene. These proteins replace the transpeptidase PBP2, an enzyme involved in bacterial cell wall formation. Since the alternative proteins PBP2a and PBP2c have low affinity for methicillin and penicillin, the presence of these antibiotics does not inhibit their activity, and cell wall synthesis continues normally, thus ensuring bacterial growth even in the presence of these antibiotics.

Since the appearance of the first MRSA strains, several methods for detecting this resistance property have been developed, according to three main approaches:
phenotypic methods are based on an evaluation of bacterial growth in the presence of antibiotics;
molecular biology methods are based on the detection of the MecA and/or MecC genes in the bacterial genome of the strains studied;
immunological and proteomic methods are based on the detection of the PBP2a protein or its peptides. Among these technologies, immuno-chromatography (detection using antibodies) and mass spectrometry methods are particularly used. These different approaches are briefly presented below.

### Phenotypic Methods

Traditional agar diffusion methods, although old, are still widely used in laboratories due to their simplicity and low cost. They consist of culturing the bacterium on agar in the presence of an antibiotic disc, usually oxacillin or cefoxitin. The sensitive or resistant phenotype of the bacterial strain is determined by evaluating the diameter of the growth inhibition zone around the disc.

The main disadvantage of this technology is the time it takes, approximately 12 to 18 hours, to obtain the result (Felten, 2002).

The arrival on the market of instruments such as the Vitek® 2 distributed by bioMérieux, or the Phoenix™ distributed by Becton Dickinson, has enabled the high-throughput automation of antibiotic susceptibility testing. Although more robust than agar diffusion methods, these tests are also limited by the growth time of bacteria. In the case of Staphylococcus aureus, the result is obtained in about 10 to 13 hours.

For example, the article by Sparbier et al., 2013, discloses such a phenotypic method where bacterial growth is evaluated in the presence of antibiotics. Bacteria are incubated in the presence of oxacillin or cefoxitin, and high molecular weight “heavy” lysine. Incorporation of this heavy amino acid will only be observed in newly synthesized proteins. However, only bacteria resistant to antibiotics exhibit significant protein biosynthesis activity. The proteins comprising the heavy lysine are then detected by MALDI-TOF.

The major disadvantage of this so-called phenotypic technology is that it is sensitive to factors that can influence bacterial growth, such as inoculum volume, incubation time and temperature, medium, pH or salt concentration (Tenover et al., 1999).

### Molecular Biology Methods

The advent of molecular biology methods has significantly improved the identification times of MRSA strains compared to phenotypic methods. The high sensitivity and specificity of the tests currently on the market make the detection of the MecA gene by polymerase chain reaction (PCR) the gold standard for the diagnosis of MRSA. Commonly used tests include GeneXpert MRSA/SA BC distributed by Cepheid, GeneOhm™ StaphSR Assay distributed by Becton Dickinson, eazyplex® MRSAplus distributed by AmplexDiagnostics or FilmArray BCID distributed by bioMerieux. These tests allow the detection of MRSA strains in 1 to 3 hours, directly from a positive blood culture bottle, without the need for prior subculture steps consisting of isolating the bacteria and causing them to proliferate.

The main disadvantage of this technology is that it tends to generate false positives, especially in the case of strains carrying an SSCmec cassette that does not contain the MecA gene, or for strains expressing a variant protein encoded by a gene having a sequence slightly different from that established for the MecA gene.

Moreover, the high cost of molecular biology analysis remains a major obstacle to their routine use.

### Immunological Tests: Detection of PBP2A Protein

The use of latex beads coupled with a specific monoclonal antibody detecting the PBP2a protein makes it possible to see agglutination with the naked eye, indicating the presence of the antigen in the sample. This is the principle on which the Oxoid PBP2′ test distributed by Thermo Fisher Scientific or the Mastalex-MRSA test distributed by Mast Diagnostic are based.

The duration of the test itself is comprised between 15 and 20 minutes. This test performs very well, with a sensitivity of 100% and a specificity greater than 99%.

The major disadvantage of this technique is that it requires a minimum quantity of 1.5×10⁹ bacteria in the sample tested, which involves a bacterial culture step lasting at least 10 hours.

Moreover, these tests do not allow the detection of MRSA strains expressing the PBP2c protein, which is not recognized by certain monoclonal antibodies used. This has especially been shown for the Oxoid PBP2′ test (Dupieux et al., 2017).

Immunochromatographic tests have also been marketed for the detection of MRSA strains, for example the Clearview™ PBP2a SA Culture Colony Test kit distributed by Alere.

Although very fast and possessing a sensitivity and specificity greater than 98%, this test also requires a bacterial culture step.

The Clearview™ PBP2a SA Culture Colony Test can advantageously detect PBP2c from colonies growing on the edge of a cefoxitin disc after agar culture. However, this induction step necessary for the detection of MecC results in an additional delay of approximately 18 hours before the result is rendered (Dupieux et al., 2017).

### Targeted Mass Spectrometry

Recent studies have highlighted the potential of so-called bottom-up proteomic approaches, i.e. based on targeted mass spectrometry peptide fragmentation spectra, for the identification of pathogens and the demonstration of resistance mechanisms.

These approaches include the following steps:
1) mechanical or chemical lysis of the bacterial sample to release intracellular proteins;
2) enzymatic digestion of bacterial proteins using proteases (typically trypsin) to generate a mixture of peptides;
3) chromatographic separation coupled with targeted mass spectrometry analysis of the sample, where the mass spectrometer operates in MS/MS mode for the detection of marker peptides of resistance proteins;
4) reprocessing of the results to validate the presence/absence of marker peptides in the sample.

The term “targeted” indicates that specific peptide sequences are sought; these can be whole protein sequences or peptides derived from enzymatic digestion of said proteins.

Targeted mass spectrometry has been successfully used to characterize the resistance and virulence of Staphylococcus aureus strains by especially detecting the PBP2a protein or virulence factors such as PVL (Charretier et al., 2015).

International application WO 2011/045544 describes a method comprising the characterization of the resistance and virulence of Staphylococcus aureus by liquid chromatography coupled with targeted mass spectrometry, in which the starting biological material is a colony isolated on agar medium.

Finally, it has recently been shown that the PBP2a protein can be detected in its intact form, after separation by liquid chromatography and MS analysis (top-down technology based on analysis of the fragmentation spectra of whole proteins). In the absence of the enzymatic protein digestion step, the time required to obtain a result is shortened (Neil et al., 2021). However, the authors did not demonstrate the applicability of their method to the detection of PBP2a directly in blood culture samples without a prior subculture step.

To date, no technology meeting all the criteria required for hospital installation, especially short analysis time, simplicity of sample preparation, limited cost and satisfactory performance, is known.

In order to satisfy the current needs of analytical laboratories, a method for identifying MRSA strains must meet at least the following criteria:
no subculture step of bacteria isolated from the blood (analysis directly from the blood culture bottle when it is detected as positive);
short sample preparation and analysis time (less than 2 hours) in order to obtain a result quickly, and thus allow the administration to the patient of an appropriate antibiotic treatment in the shortest possible time;
low cost so that the test can be carried out routinely;
test detecting both proteins PBP2a and PBP2c with a sensitivity and specificity close to 100%, including variant proteins having a peptide sequence having sequence identity to SEQ ID NO. 1 or 2.

The inventors have developed a targeted method for detecting MRSA strains by liquid chromatography coupled with mass spectrometry, carried out directly from a positive blood culture bottle, including an induction step and rapid sample preparation, and allowing a result to be obtained in less than 1.5 h from the detection of the presence of a strain of Staphylococcus aureus in a biological sample.

### DISCLOSURE OF THE INVENTION

The present invention concerns a method for determining the methicillin resistance properties of a strain of Staphylococcus aureus present in a biological sample, comprising the following steps:
a) incubating the biological sample containing said S. aureus strain for at least 15 minutes in the presence of an antibiotic from the beta-lactam class chosen from the following group: cefoxitin and 6-aminopenicillanic acid (6-APA);
b) isolating the bacteria present in said biological sample;
c) lysing the bacteria and hydrolysing the bacterial proteins in order to obtain a mixture of peptides;
d) analysing this mixture of peptides by targeted mass spectrometry, the detection of at least one peptide derived from the PBP2a (SEQ ID NO. 1) or PBP2c (SEQ ID NO. 2) proteins during this analysis step being indicative of the methicillin resistance of the Staphylococcus aureus strain present in said biological sample.

The present invention also concerns a kit for carrying out this method, comprising:
an antibiotic of the beta-lactam class chosen from the following group: cefoxitin and 6-aminopenicillanic acid (6-APA);
trypsin;
a reagent allowing selective lysis of non-bacterial cells present in the biological sample, in particular a detergent chosen from saponin, Triton X100 and sodium dodecyl sulfate (SDS);
optionally, reagents for targeted mass spectrometry.

### DESCRIPTION OF THE FIGURES

FIG. 1 shows a chromatogram resulting from an MRM analysis of the MRSA26b strain. A) Sample prepared without the induction step. B) Sample prepared with the induction step. The peaks corresponding to the transitions of the PBP2a peptides are indicated by arrows.

FIG. 2 shows chromatograms resulting from an MRM3 analysis of the MRSA26b strain. A1 and A2) MRM3 chromatograms of the VALELGSK (A1) and FQITTSPGSTQKK (A2) peptides for the sample prepared without the induction step. B1 and B2) MRM3 chromatograms of the VALELGSK (B1) and FQITTSPGSTQKK (B2) peptides for the sample prepared with the induction step.

FIG. 3 shows a chromatogram resulting from an MRM analysis of the MRSA28b strain. A) Sample prepared without the induction step. B) Sample prepared with the induction step. The peaks corresponding to the transitions of the PBP2a peptides are indicated by arrows.

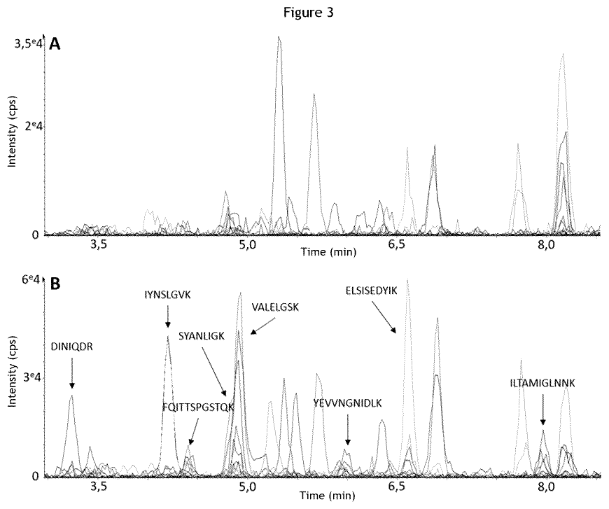

FIG. 4 shows chromatograms resulting from an MRM3 analysis of the MRSA28b strain. A1 and A2) MRM3 chromatograms of the VALELGSK (A1) and FQITTSPGSTQKK (A2) peptides for the sample prepared without the induction step. B1 and B2) MRM3 chromatograms of the VALELGSK (B1) and FQITTSPGSTOKK (B2) peptides for the sample prepared with the induction step.

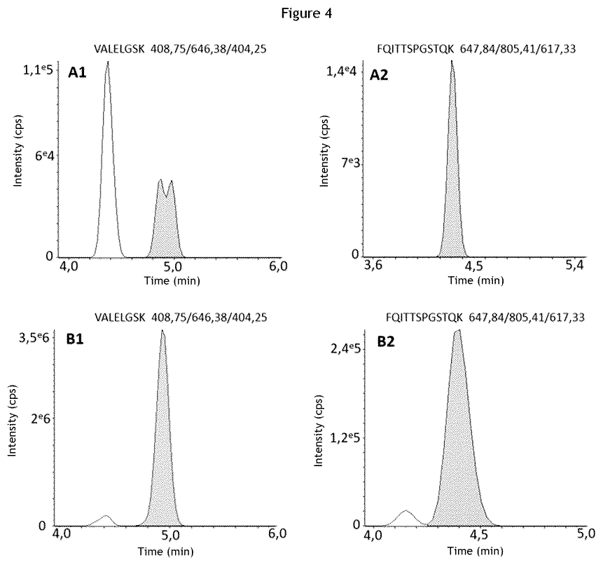

FIG. 5 shows chromatograms of the MSSA16b strain after an induction step. A) MRM chromatograms. B1 and B2) MRM3 chromatograms for the VALELGSK (B1) and FQITTSPGSTOKK (B2) peptides. No peptides are detected for both types of analysis.

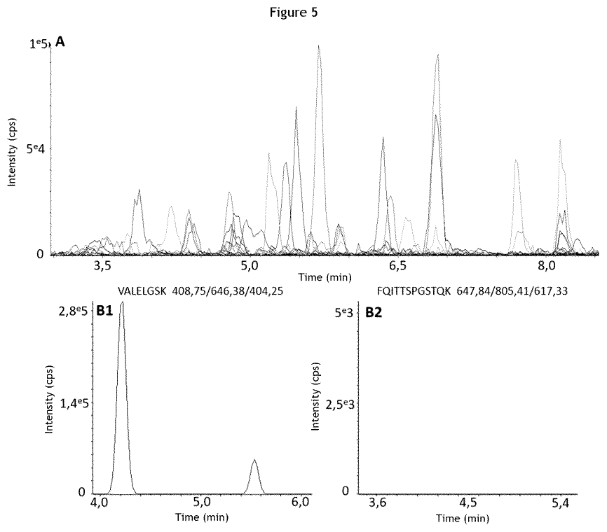

FIG. 6 shows chromatograms of the MSSA1b strain after an induction step. A) MRM chromatograms. B1 and B2) MRM3 chromatograms for the VALELGSK (B1) and FQITTSPGSTQkk (B2) peptides. No peptides are detected for both types of analysis.

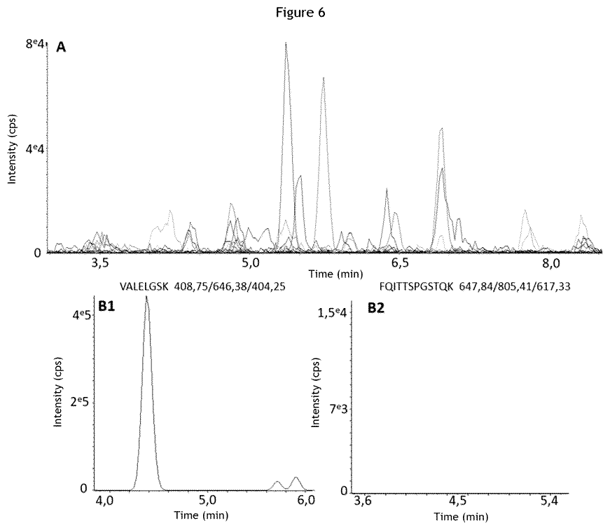

FIG. 7 shows a chromatogram resulting from MRM analysis of a strain expressing PBP2c. A) Sample prepared without the induction step. B) Sample prepared with the induction step. The peaks corresponding to the transitions of the PBP2c peptides are indicated by arrows.

### DETAILED DESCRIPTION OF THE INVENTION

The present invention concerns a method for detecting methicillin-resistant strains of Staphylococcus aureus, called MRSA strains, making it possible to obtain a result in less than 1.5 h from a biological sample identified as containing a strain of S. aureus.

Methicillin, also spelled meticillin, is a beta-lactam antibiotic belonging to the penicillin subfamily. Its CAS number is 61-32-5. It has been widely used against Staphylococcus aureus infections, before being supplanted by cloxacillin, which is less likely to develop bacterial resistance.

Advantageously, the method of the invention makes it possible to identify strains resistant to this antibiotic (MRSA) with a sensitivity and specificity close to 100%.

The method according to the invention uses a biological sample without a bacterial subculture step, for example a positive blood culture sample (containing blood cells and bacteria). A first step of induction of expression of the PBP2a or PBP2c protein, using an antibiotic of the beta-lactam class, is followed by a step of rapid isolation of the bacteria, then a step of lysis of the bacteria and enzymatic digestion of the proteins, and finally, targeted mass spectrometry analysis for the detection of peptides from the enzymatic digestion of the PBP2a or PBP2c protein.

Thus, the present invention concerns a method for determining the methicillin resistance properties of a strain of Staphylococcus aureus present in a biological sample, comprising the following steps:
a) incubating the biological sample containing said Staphylococcus aureus strain for at least 15 minutes in the presence of an antibiotic from the beta-lactam class chosen from the following group: cefoxitin and 6-aminopenicillanic acid (6-APA);
b) isolating the bacteria present in said biological sample;
c) lysing bacteria and hydrolysing the bacterial proteins, in order to obtain a mixture of peptides;
d) analysing this mixture of peptides by targeted mass spectrometry; the detection of at least one peptide derived from the PBP2a protein or PBP2c protein in this analysis step is indicative of the resistance to methicillin of the strain of Staphylococcus aureus present in said biological sample.

This method was developed from the method described in international application WO 2011/045544.

On the basis of this method, the inventors have identified that the PBP2a protein is expressed very heterogeneously among MRSA strains, at very different levels of expression.

Using a methodology similar to that presented in application WO 2011/045544, but with as starting biological sample a blood culture medium positive for Staphylococcus aureus, i.e., without a bacterial subculture step, it has been demonstrated on a cohort of 98 MRSA strains representative of French epidemiology that approximately 60% of said MRSA strains have a PBP2a expression level that is too low to be detected by the methodology described (see experimental part).

In fact, there is heterogeneity of PBP2a expression between different MRSA strains. Some strains naturally express PBP2a at high levels (detectable without induction) and others have very low basal expression levels, which do not allow detection of PBP2a by direct analysis without an induction step.

It was therefore concluded that the technology described in application WO 2011/045544 is not suitable for implementation in a clinical analysis laboratory, because many MRSA strains cannot be detected.

The present application concerns an improvement of said method, comprising the addition of a step (a) of incubating the biological sample containing said S. aureus strain for at least 15 minutes in the presence of a beta-lactam antibiotic, to induce the expression of the PBP2a protein or the PBP2c protein and thus have a new expression sufficient to detect the variant protein expressed in 100% of the MRSA strains.

The PBP2a protein has the following sequence:

Sequence 1, PBP2a, Staphylococcus aureus (NCBI ID: WP_001801873.1):

MMKKIKIVPLILIVVVVGFGIYFYASKDKEINNTIDAIEDKNFKQVYKDS
SYISKSDNGEVEMTERPIKIYNSLGVKDINIQDRKIKKVSKNKKRVDAQY
KIKTNYGNIDRNVQFNFVKEDGMWKLDWDHSVIIPGMQKDQSIHIENLKS
ERGKILDRNNVELANTGTAYEIGIVPKNVSKKDYKAIAKELSISEDYIKQ
QMDQNWVQDDTFVPLKTVKKMDEYLSDFAKKFHLTTNETESRNYPLGKAT
SHLLGYVGPINSEELKQKEYKGYKDDAVIGKKGLEKLYDKKLQHEDGYRV
TIVDDNSNTIAHTLIEKKKKDGKDIQLTIDAKVQKSIYNNMKNDYGSGTA
IHPQTGELLALVSTPSYDVYPFMYGMSNEEYNKLTEDKKEPLLNKFQITT
SPGSTQKILTAMIGLNNKTLDDKTSYKIDGKGWQKDKSWGGYNVTRYEVV
NGNIDLKQAIESSDNIFFARVALELGSKKFEKGMKKLGVGEDIPSDYPFY
NAQISNKNLDNEILLADSGYGQGEILINPVQILSIYSALENNGNINAPHL
LKDTKNKVWKKNIISKENINLLTDGMQQVVNKTHKEDIYRSYANLIGKSG
TAELKMKQGETGRQIGWFISYDKDNPNMMMAINVKDVQDKGMASYNAKIS
GKVYDELYENGNKKYDIDE

The PBP2c protein has the following sequence:

Sequence 2, PBP2c, Staphylococcus aureus (NCBI ID: WP_000725529.1):

MKKIYISVLVLLLIMIIITWLFKDDDIEKTISSIEKGNYNEVYKNSSEKS
KLAYGEEEIVDRNKKIYKDLSVNNLKITNHEIKKTGKDKKQVDVKYNIYT
KYGTIRRNTQLNFIYEDKHWKLDWRPDVIVPGLKNGQKINIETLKSERGK
IKDRNGIELAKTGNTYEIGIVPNKTPKEKYDDIARDLQIDTKAITNKVNQ
KWVQPDSFVPIKKINKQDEYIDKLIKSYNLQINTIKSRVYPLNEATVHLL
GYVGPINSDELKSKQFRNYSKNTVIGKKGLERLYDKQLQNTDGFKVSIAN
TYDNKPLDTLLEKKAENGKDLHLTIDARVQESIYKHMKNDDGSGTALQPK
TGEILALVSTPSYDVYPFMNGLSNNDYRKLTNNKKEPLLNKFQITTSPGS
TQKILTSIIALKENKLDKNTNFDIYGKGWQKDASWGNYNITRFKVVDGNI
DLKQAIESSDNIFFARIALALGAKKFEQGMQDLGIGENIPSDYPFYKAQI
SNSNLKNEILLADSGYGQGEILVNPIQILSIYSALENNGNIQNPHVLRKT
KSQIWKKDIIPKKDIDILTNGMERVVNKTHRDDIYKNYARIIGKSGTAEL
KMNQGETGRQIGWFVSYNKNNPNMLMAINVKDVQNKGMASYNATISGKVY
DDLYDNGKTQFDIDQ

Advantageously, the method of the present invention makes it possible to detect the two PBP2a and PBP2c proteins as well as variant proteins.

The term “variant protein” is understood to mean a protein having a peptide sequence having a strong sequence identity with the sequences SEQ ID NO. 1 or 2, in particular a sequence identity of at least 90%, or better still of at least 95%, or even of 99% with one of the sequences SEQ ID NO. 1 or SEQ ID NO. 2. Variant proteins generally have one, two or three point mutations in the wild-type protein sequences, i.e. they differ only for one, two or three amino acids in the peptide sequence.

The percentages of identity to which reference is made in the context of the disclosure of the present invention are determined after optimal alignment of the sequences to be compared, which can therefore comprise one or more additions, deletions, truncations and/or substitutions.

This percentage of identity can be calculated by any sequence analysis method well known to the person skilled in the art.

The percentage of identity can be determined after global alignment of the sequences to be compared taken in their entirety, over their entire length. In addition to the manual method, it is possible to determine the global alignment of the sequences by means of the algorithm of Needleman and Wunsch (1970).

For amino acid sequences, the sequences can be compared using any software well known to the person skilled in the art, such as, for example, Needle software. The parameters used can especially include: “Gap Open” equal to 10.0, “Gap Extend” equal to 0.5 and the BLOSUM62 matrix.

Preferably, the percentage of identity defined in the context of the present invention is determined by means of a global alignment of the sequences to be compared over their entire length.

### Step (a) of Inducing the Expression of PBP2a or PBP2c

Induction consists of carrying out a rapid incubation (in particular, lasting less than 3 hours and preferably less than 1 hour) in the presence of an antibiotic in order to activate the systems regulating the expression of PBP2a or PBP2c proteins and thus lead to an overexpression of these proteins.

This incubation of the biological sample containing said strain of Staphylococcus aureus for at least 15 minutes, in the presence of an antibiotic of the beta-lactam class chosen from cefoxitin and 6-aminopenicillanic acid (6-APA), induces the expression of at least one protein chosen from PBP2a and PBP2b in said bacterial strain.

The antibiotic used is of the beta-lactam class, i.e. an antibiotic containing a beta-lactam ring. This antibiotic is chosen from cefoxitin and 6-aminopenicillanic acid (6-APA).

Cefoxitin, CAS number 35607-66-0, is an antibiotic of the beta-lactam family, classified among the so-called second generation cephalosporins. Cefoxitin exerts a bactericidal action by inhibiting cell wall synthesis.

6-aminopenicillanic acid, abbreviated 6-APA, CAS number 551-16-6, is a derivative of penicillin.

Preferably, the antibiotic used for induction step (a) is cefoxitin.

The induction of PBP2a or PBP2c expression is carried out by incubation of the biological sample in the presence of an appropriate amount of antibiotic, typically at a temperature comprised between 30° and 40 Celsius. Incubation of the biological sample will preferably take place at 37° C., with stirring.

According to a particular implementation of the method of the invention, the incubation step (a) is carried out for a period of at least 15 minutes.

Preferably, this incubation step (a) is carried out for a period of less than 3 hours, or less than 2 hours, or preferentially less than 1 hour.

Advantageously, the incubation time may be at least 15 minutes, at least 20 minutes, at least 25 minutes, at least 30 minutes, at least 35 minutes, at least 40 minutes or at least 45minutes.

In particular, this incubation step (a) is carried out for one of the following durations:
15 to 180 minutes;
15 to 120 minutes;
15 to 90 minutes; or
15 to 60 minutes.

As shown in the examples, in the absence of this step of inducing expression of PBP2a or PBP2c, some methicillin-resistant S. aureus strains are not identified as such, because the detection of one of the PBP2a or PBP2c proteins is impossible because of the too low expression of said protein in said strains.

### Step (b) of Isolating the Bacteria

The bacteria can be isolated by any means known to the person skilled in the art, in particular by centrifugation.

According to one implementation of the method, step (b) also comprises a step of selective lysis of the non-bacterial cells present in the biological sample, this selective lysis step being carried out before centrifugation of the sample.

Detergent compounds allow lysis of animal cells by dissociation of the membrane. Since bacteria are composed of a rigid peptidoglycan wall, they are not lysed by the action of detergent.

A detergent compound may be selected from the group of saponin, Triton X100 or sodium dodecyl sulfate (SDS), for example.

In particular, when the biological sample is a blood sample, step (b) of isolating the bacteria is advantageously carried out concomitantly with lysis of the blood cells present in the sample, by means of the addition of a detergent compound.

### Step (c) of Lysing the Bacteria and Hydrolysing the Bacterial Proteins

Various methods for lysing bacterial cells can be used. Examples include thermal methods (temperature above 100° C. for 10 minutes, or freezing in liquid nitrogen), enzymatic methods (action of lysozyme or lyticase) or mechanical methods (high pressure, grinding or sonication).

According to a preferred implementation of the method of the invention, the bacteria are lysed by sonication.

The hydrolysis of bacterial proteins into peptides is usually carried out by one of the following two processes:
a) by the action of chemical agents such as hydroxyl radicals which induce random cleavages at the level of peptide bonds; or
b) by the action of proteolytic enzymes (proteases) which have an action of hydrolysis of protein bonds.

According to a preferred implementation of the method, the hydrolysis of bacterial proteins in step (c) is enzymatic hydrolysis.

The proteases commonly used include pepsin, which hydrolyses peptide bonds preferentially before aromatic amino acids (tyrosine, tryptophan and phenylalanine), GluC endoproteinase, which cleaves peptide bonds at glutamate residues, or trypsin, which cleaves proteins on the C-terminal side of the amino acids lysine and arginine.

According to a preferred implementation of the method of the invention, trypsin is used as enzyme for the bacterial protein hydrolysis step. Trypsin is preferred for the specific nature of its activity, the appropriate size of the peptides it generates, and the nature of the tryptic peptides which possess on the C-terminal side an amino acid that can be positively charged (lysine or arginine), thus facilitating analysis by mass spectrometry (analysis of charged molecules).

A specific protocol for step (c) is presented in the experimental part.

### Step (d) of Analysis by Targeted Mass Spectrometry

Mass spectrometry is a physical analysis technology that detects and identifies molecules of interest. It is also known as single reaction monitoring, or multiple reaction monitoring, or parallel reaction monitoring. Its principle lies in the gas phase separation of charged molecules (ions) according to their mass/charge ratio (m/z).

Mass spectrometers comprise:
i) an ionization source for ionizing the molecules to be analysed, i.e. conferring a positive or negative charge to these molecules;
ii) a mass analyser for separating the ionized molecules according to their mass to charge ratio (m/z);
iii) a detector for measuring the signal produced either directly by the molecular ions or by ions produced from the molecular ions, as described below.

The ionization step necessary for carrying out mass spectrometry can be carried out by any method known to the person skilled in the art. The ionization source makes it possible to bring the molecules to be assayed into a gaseous and ionized state. An ionization source can be used either in positive mode to study positive ions or in negative mode to study negative ions. Several types of sources exist and will be used depending on the desired result and the molecules analysed.

The mass analyser in which the step of separating the ionized markers as a function of their mass/charge ratio (m/z) is carried out is any mass analyser known to the person skilled in the art. Examples include low-resolution analysers, such as quadrupole (Q), 3D ion trap (IT) or linear ion trap (LIT), and high-resolution analysers, which measure the exact mass of analytes and which use the magnetic sector coupled to an electrical sector, the time of flight (TOF), or Orbitrap.

The separation of the molecular ions as a function of their m/z ratio can be carried out once (simple mass spectrometry or MS), or several successive MS separations can be carried out. When two successive MS separations are performed, the analysis is called MS/MS or MS2. When three successive MS separations are performed, the analysis is called MS/MS/MS or MS3.

Targeted mass spectrometry is a variant in which the molecules of interest sought by this analytical technique are known beforehand, and the analysis is used to identify whether they are present in a sample.

Targeted approaches (multiple reaction monitoring, MRM; parallel reaction monitoring, PRM; multiple reaction monitoring-high resolution, MRM-HR; multiple reaction monitoring cubed, MRM3, DIA/SWATH) consist of the selection by a first analyser of a precise mass corresponding to the peptide of interest which is then fragmented in a collision cell. The fragments generated are then monitored by a third analyser and their signal/intensity is measured.

In the case of chromatographic couplings, the signal of the fragments is measured as a function of time to be represented in the form of a chromatogram, the appearance of concomitant chromatographic peaks (simultaneous detection of the fragments) thus being evidence of the presence of the peptide in the sample.

These approaches require preliminary selection and validation of marker peptides to ensure their sequence/mass uniqueness (or mass/charge ratio, m/z) and their detectability.

The principle of the SRM mode, or of the MRM mode, is to specifically select a precursor ion, to fragment it, and then to specifically select one of its fragment ions. For such applications, devices of the triple quadrupole type or triple quadrupole hybrids with an ion trap are generally used.

The DIA/SWATH analysis mode consists of recording the fragmentation spectra of contiguous precursor ion selection windows, usually overlapping by 1 unit of mass-to-charge ratio (m/z), the windows being characterized by a fixed or variable width in m/z, or by using a sliding window of fixed width in m/z, while ensuring that the total cycle time to cover all these windows allows each chromatographic peak to be sampled by at least 5 points.

According to a preferred embodiment of the invention, mass spectrometry analysis is targeted during the analysis (SRM/MRM, MRM, MRM3 or PRM mode) or after the analysis (DIA/SWATH mode).

More preferably, the analysis by targeted mass spectrometry is carried out in MRM or MRM3 mode, and most preferably is carried out in MRM3 mode.

The targeted mass spectrometry is coupled with separation of the peptides, preferably by chromatographic or electrophoretic separation of the peptides.

The separation of the peptides can be carried out by any technique known to the person skilled in the art, and in particular can be carried out by reversed phase liquid chromatography, by normal phase liquid chromatography, by hydrophilic phase liquid chromatography, or by capillary electrophoresis.

Preferably, the separation of the peptides is carried out by reversed-phase liquid chromatography.

The peptides sought during this analysis step are peptides derived from the PBP2a or PBP2c proteins, specific for these proteins.

It is understood that at least one peptide derived from these proteins can be detected, but that preferably several peptides will be detected during the analysis, which confirms the results obtained.

According to a preferred implementation of the method of the invention, the at least one peptide derived from the PBP2a or PBP2c protein that is detected, i.e., whose presence is detected by targeted mass spectrometry, is chosen from the group of 12 peptides having the following sequences: SEQ ID NO. 3 to SEQ ID NO. 14 presented in Tables 1 and 2 below.

TABLE 1
Peptide sequences derived from PBP2a

| SEQ | | |
|---|---|---|
| ID | Peptide derived | Location in the |
| NO. | from PBP2a | sequence SEQ ID NO. 1 |
| 3 | IYNSLGVK | 70-77 |
| 4 | DINIQDR | 78-84 |
| 5 | ELSISEDYIK | 190-199 |
| 6 | FQITTSPGSTQK | 396-407 |
| 7 | ILTAMIGLNNK | 408-418 |
| 8 | YEVVNGNIDLK | 447-457 |
| 9 | VALELGSK | 471-478 |
| 10 | SYANLIGK | 591-598 |

TABLE 2
Peptide sequences derived from PBP2c

| SEQ | Peptide derived | Location in the |
|---|---|---|
| ID NO. | from PBP2c | sequence SEQ ID NO. 2 |
| 11 | LAYGEEEIVDR | 52-62 |
| 12 | SYNLQINTIK | 227-236 |
| 13 | ILTSIIALK | 404-412 |
| 14 | IALALGAK | 467-474 |

Advantageously, all these peptides have been selected in conserved regions of the proteins and are therefore also identified in the variant proteins of PBP2a and PBP2c.

The method according to the invention may be carried out on any type of biological sample capable of containing a Staphylococcus aureus strain.

According to a particular implementation of the method, the biological sample is chosen from:
a biological fluid, for example blood, serum, plasma, urine, cerebrospinal fluid, and tears;
a bacterial culture, for example a blood culture, a bacterial colony on agar, a bacterial culture broth;
a food sample; and
any other type of biological sample.

It will preferably be a biological fluid, in particular a blood sample (blood, serum, plasma), and more particularly the medium of a positive blood culture containing a Staphylococcus aureus.

The term “blood culture” is understood to mean a blood sample taken from a patient and then incubated under suitable conditions to allow proliferation of any bacteria present in said sample.

This blood culture can be carried out in blood culture bottles such as those marketed in the Bact/Alert range distributed by bioMerieux, or those of the Bactec range distributed by Becton Dickinson.

### Kit for Implementing the Method

The present invention also concerns a kit for implementing the method as described above, comprising:
an antibiotic of the beta-lactam class chosen from the following group: cefoxitin and 6-aminopenicillanic acid (6-APA);
trypsin;
a reagent allowing selective lysis of non-bacterial cells present in the biological sample, for example a detergent.

For example, the reagents allowing selective lysis of non-bacterial cells include a detergent compound selected from the following group of compounds: saponin, Triton X100 or sodium dodecyl sulfate (SDS).

### EXAMPLES

The examples presented below are intended to illustrate the method according to the invention, but are in no way a limitation of the object of the invention.

In particular, in the examples presented below, the antibiotic used during the induction step is cefoxitin, but it is understood that 6-APA may also be used.

### Example 1. Material and Method

Step (a) of induction is carried out according to the following protocol:
Disinfect the septum of the blood culture bottle.
Using a 21G syringe and needle, draw off 3.8 mL of positive blood culture medium and transfer to a 15 ml tube.
Add 200 μL of 8 μg/mL cefoxitin solution.
Homogenize the mixture and incubate the tube at 37° C. with stirring (180 rpm) for 30 minutes.

Step (b) of isolating bacteria is advantageously carried out concomitantly with lysis of blood cells, according to the following protocol:
After 30 minutes of induction, transfer 1 mL of medium to a 1.5 ml tube and add 200 μL of 12% SDS solution and vortex for 10 seconds.
Centrifuge for 2 minutes at 16, 100 g and remove the supernatant.
Resuspend the pellet in 1 mL of saline.
Centrifuge for 1 minute at 16,100 g and discard the supernatant.
Resuspend in 1 mL saline.

Step (c) of mechanical lysis of bacteria and enzymatic digestion of proteins is carried out according to the following protocol:
Transfer 200 μL of the previously prepared bacterial suspension to a 1.5 mL Eppendorf LoBind tube containing approximately 70 mg of glass beads (Glass beads, acid-washed, 150-212 μm, Sigma-Aldrich, ref G1145).
Add 50 μL of a 1 mg/ml trypsin solution prepared extemporaneously in 150 mm ammonium bicarbonate buffer from lyophilized trypsin.
Immediately place the sample in the water bath of a sonicator set at 50° C. and then start 10 ultrasound cycles (low power).
   30 seconds ultrasound on
   30 seconds ultrasound off
Directly at the end of the 10 ultrasound cycles, add 5 μL of formic acid
Centrifuge the tube at 9600 g for 5 minutes.
Transfer 150 μL of supernatant to a 2 mL amber glass vial with insert for mass spectrometry analysis.

Step (d) of targeted mass spectrometry analysis is carried out according to the following protocol:

A volume of 5 μL of sample from the previous lysis/digestion step is injected into the chromatographic system. The analysis is performed on a Waters XBridge Peptide BEH C18 reversed-phase column, inner diameter 1 mm, length 100 mm, particle size 3.5 μm, pore size 130 Å using a chromatographic system with an Agilent 1290 infinity LC pump, an Agilent 1290 Autosampler and an Agilent 1290 TCC column oven set to 60° C. The gradient used for the chromatographic separation is presented in Table 3.

Solvent A: H2O+0.1% formic acid

Solvent B: Acetonitrile+0.1% formic acid

TABLE 3
Analysis parameters

| Time | Flow rate | Solvent A | Solvent B |
|---|---|---|---|
| (min) | (μL/min) | (%) | (%) |
| 0 | 75 | 98 | 2 |
| 0.5 | 75 | 90 | 10 |
| 15.5 | 75 | 60 | 40 |
| 15.7 | 150 | 25 | 75 |
| 17 | 150 | 25 | 75 |
| 17.1 | 150 | 98 | 2 |
| 21.2 | 150 | 98 | 2 |
| 21.3 | 75 | 98 | 2 |
| 22 | 75 | 98 | 2 |

The outlet of the chromatographic system is directly connected to the ionization source of a QTRAP 6500+ mass spectrometer (Sciex) for on-line analysis of peptides from bacterial protein digestion. The mass spectrometer is operated in MRM or MRM-cubed (MRM3) mode and the transitions monitored, for the PBP2a protein, are described in Table 4.

TABLE 4
List of transitions monitored for PBP2a detection.

| | | | | | Charge | | | |
|---|---|---|---|---|---|---|---|---|
| | | | | | state | Fragment | De- | |
| | m/z | m/z | Dwell | | of the | ion | clustering | Collision |
| Transition | filtered | filtered | time | | pre- | (un- | potential | Energy |
| number | in Q1 | in Q3 | (ms) | Peptide | cursor | charged) | (V) | (V) |
| 1 | 437.225 | 645.331 | 20 | DINIQDR | 2 | y5 | 43 | 24.6 |
| 2 | 437.225 | 531.289 | 20 | DINIQDR | 2 | y4 | 43 | 24.6 |
| 3 | 437.225 | 418.204 | 20 | DINIQDR | 2 | y3 | 43 | 24.6 |
| 4 | 594.344 | 961.514 | 20 | ILTAMIGLNNK | 2 | y9 | 47.3 | 30.3 |
| 5 | 594.344 | 860.466 | 20 | ILTAMIGLNNK | 2 | y8 | 47.3 | 30.3 |
| 6 | 594.344 | 789.429 | 20 | ILTAMIGLNNK | 2 | y7 | 47.3 | 30.3 |
| 7 | 594.344 | 658.388 | 20 | ILTAMIGLNNK | 2 | y6 | 47.3 | 30.3 |
| 8 | 594.344 | 545.304 | 20 | ILTAMIGLNNK | 2 | y5 | 47.3 | 30.3 |
| 9 | 598.806 | 954.478 | 20 | ELSISEDYIK | 2 | y8 | 47.5 | 30.4 |
| 10 | 598.806 | 867.446 | 20 | ELSISEDYIK | 2 | y7 | 47.5 | 30.4 |
| 11 | 598.806 | 754.362 | 20 | ELSISEDYIK | 2 | y6 | 47.5 | 30.4 |
| 12 | 598.806 | 667.33 | 20 | ELSISEDYIK | 2 | y5 | 47.5 | 30.4 |
| 13 | 647.836 | 1019.537 | 20 | FQITTSPGSTQK | 2 | y10 | 90 | 31 |
| 14 | 647.836 | 906.453 | 20 | FQITTSPGSTQK | 2 | y9 | 90 | 31 |
| 15 | 647.836 | 805.405 | 20 | FQITTSPGSTQK | 2 | y8 | 90 | 31 |
| 16 | 647.836 | 704.357 | 20 | FQITTSPGSTQK | 2 | y7 | 90 | 31 |
| 17 | 647.836 | 617.325 | 20 | FQITTSPGSTQK | 2 | y6 | 90 | 31 |
| 18 | 447.258 | 780.425 | 20 | IYNSLGVK | 2 | y7 | 43.3 | 25 |
| 19 | 447.258 | 617.362 | 20 | IYNSLGVK | 2 | y6 | 43.3 | 25 |
| 20 | 447.258 | 503.319 | 20 | IYNSLGVK | 2 | y5 | 43.3 | 25 |
| 21 | 447.258 | 416.287 | 20 | IYNSLGVK | 2 | y4 | 43.3 | 25 |
| 22 | 433.243 | 778.446 | 20 | SYANLIGK | 2 | y7 | 42.9 | 24.5 |
| 23 | 433.243 | 615.382 | 20 | SYANLIGK | 2 | y6 | 42.9 | 24.5 |
| 24 | 433.243 | 544.345 | 20 | SYANLIGK | 2 | y5 | 42.9 | 24.5 |
| 25 | 408.745 | 717.414 | 20 | VALELGSK | 2 | y7 | 20 | 20 |
| 26 | 408.745 | 646.377 | 20 | VALELGSK | 2 | y6 | 20 | 20 |
| 27 | 408.745 | 533.293 | 20 | VALELGSK | 2 | y5 | 20 | 20 |
| 28 | 408.745 | 404.25 | 20 | VALELGSK | 2 | y4 | 20 | 20 |
| 29 | 632.333 | 971.552 | 20 | YEVVNGNIDLK | 2 | y9 | 48.4 | 31.6 |
| 30 | 632.333 | 872.484 | 20 | YEVVNGNIDLK | 2 | y8 | 48.4 | 31.6 |
| 31 | 632.333 | 773.415 | 20 | YEVVNGNIDLK | 2 | y7 | 48.4 | 31.6 |
| 32 | 632.333 | 659.372 | 20 | YEVVNGNIDLK | 2 | y6 | 48.4 | 31.6 |

The transitions followed for the PBP2c protein are described in Table 5.

TABLE 5
List of transitions monitored for PBP2c detection

| | | | | | Charge | | De- | |
|---|---|---|---|---|---|---|---|---|
| | m/z | m/z | Dwell | | state of | Fragment | clustering | Collision |
| Transition | filtered | filtered | time | | the | ion | potential | Energy |
| number | in Q1 | in Q3 | (ms) | Peptide | precursor | (uncharged) | (V) | (V) |
| 43 | 647.320 | 946.448 | 20 | LAYGEEEIVDR | 2 | y8 | 48.8 | 32.2 |
| 44 | 647.320 | 889.426 | 20 | LAYGEEEIVDR | 2 | y7 | 48.8 | 32.2 |
| 45 | 647.320 | 760.384 | 20 | LAYGEEEIVDR | 2 | y6 | 48.8 | 32.2 |
| 46 | 647.320 | 631.341 | 20 | LAYGEEEIVDR | 2 | y5 | 48.8 | 32.2 |
| 47 | 647.320 | 502.298 | 20 | LAYGEEEIVDR | 2 | y4 | 48.8 | 32.2 |
| 48 | 597.330 | 943.557 | 20 | SYNLQINTIK | 2 | y8 | 47.4 | 30.4 |
| 49 | 597.330 | 829.514 | 20 | SYNLQINTIK | 2 | y7 | 47.4 | 30.4 |
| 50 | 597.330 | 716.430 | 20 | SYNLQINTIK | 2 | y6 | 47.4 | 30.4 |
| 51 | 597.330 | 588.372 | 20 | SYNLQINTIK | 2 | y5 | 47.4 | 30.4 |
| 52 | 597.330 | 475.287 | 20 | SYNLQINTIK | 2 | y4 | 47.4 | 30.4 |
| 53 | 597.330 | 361.245 | 20 | SYNLQINTIK | 2 | y3 | 47.4 | 30.4 |
| 54 | 486.329 | 745.482 | 20 | ILTSIIALK | 2 | y7 | 44.4 | 26.4 |
| 55 | 486.329 | 644.434 | 20 | ILTSIIALK | 2 | y6 | 44.4 | 26.4 |
| 56 | 486.329 | 557.402 | 20 | ILTSIIALK | 2 | y5 | 44.4 | 26.4 |
| 57 | 486.329 | 444.318 | 20 | ILTSIIALK | 2 | y4 | 44.4 | 26.4 |
| 58 | 486.329 | 331.234 | 20 | ILTSIIALK | 2 | y3 | 44.4 | 26.4 |
| 59 | 378.753 | 643.414 | 20 | IALALGAK | 2 | y7 | 41.5 | 22.5 |
| 60 | 378.753 | 572.377 | 20 | IALALGAK | 2 | y6 | 41.5 | 22.5 |
| 61 | 378.753 | 459.293 | 20 | IALALGAK | 2 | y5 | 41.5 | 22.5 |
| 62 | 378.753 | 388.255 | 20 | IALALGAK | 2 | y4 | 41.5 | 22.5 |
| 63 | 378.753 | 275.171 | 20 | IALALGAK | 2 | y3 | 41.5 | 22.5 |
| 64 | 378.753 | 218.150 | 20 | IALALGAK | 2 | y2 | 41.5 | 22.5 |

The mass spectrometer parameters for analysis in MRM mode are described in Table 6 below.

TABLE 6
Mass spectrometer parameters for analysis in MRM mode

| Scanning type | MRM | |
|---|---|---|
| Polarity | Positive | |
| Ionisation source | IonDrive Turbo Spray (SCIEX) | |
| Resolution Q1 | unit | |
| Resolution Q3 | unit | |
| Inter-scan pause | 5 | ms |
| Scan speed | 10 | Da/sec |
| Curtain gas | 50.00 | psi |
| Cone voltage | 5500.00 | V |
| Source temperature | 550.00° | C. |
| Nebulizer gas (GS1) | 70.00 | psi |
| Heating gas (GS2) | 60.00 | psi |
| Collision gas | high | |
| Input potential (IP) | 10.00 | V |
| Collision cell exit potential (CXP) | 12.00 | V |

| | |
|---|---|
| Software version | Analyst 1.7.2 |

The mass spectrometer parameters for analysis in MRM3 mode are described in Table 7 (peptide VALELGSK) and Table 8 (peptide FQITTSPGSTQK) below.

TABLE 7
Mass spectrometer parameters for analysis in MRM3 mode

| Scanning type | MS/MS/MS (MS3) | |
|---|---|---|
| First precursor | 408.75 | Da |
| Second precursor | 646.38 | Da |
| Centred analysis (3rd Generation Ion) | 404.25 | Da |
| Scan window | 2 | Da |
| Polarity | Positive | |
| Ionisation source | IonDrive Turbo Spray (SCIEX) | |
| Resolution Q1 | unit | |
| Resolution Q3 | LIT | |
| Fixed filling time of the linear ion trap | 200 | ms |
| Excitation time | 25 | ms |
| Entrance barrier Q3 | 8 | V |
| Inter-scan pause | 15 | ms |
| Trapping Q0 | Enabled | |
| Scan speed | 1000 | Da/sec |
| Curtain gas | 50.00 | psi |
| Cone voltage | 5500.00 | V |
| Source temperature | 550.00° | C. |
| Nebulizer gas (GS1) | 70.00 | psi |
| Heating gas (GS2) | 60.00 | psi |
| Collision gas | high | |
| Orifice potential (DP) | 20 | V |
| Collision energy (EC) | 20 | V |
| Collision energy spread (CES) | 0 | V |
| Excitation energy (AF2) | 0.1 | V |
| Input potential (IP) | 10.00 | V |
| Collision cell output potential (CXP) | 12.00 | V |

| | |
|---|---|
| Software version | Analyst 1.7.2 |

TABLE 8
Mass spectrometer parameters for analysis in MRM3 mode

| Scanning type | MS/MS/MS (MS3) | |
|---|---|---|
| First precursor | 647.84 | Da |
| Second precursor | 805.41 | Da |
| Centred analysis (3rd Generation Ion) | 617.33 | Da |
| Scan window | 2 | Da |
| Polarity | Positive | |
| Ionisation source | IonDrive Turbo Spray (SCIEX) | |
| Resolution Q1 | unit | |
| Resolution Q3 | LIT | |
| Fixed filling time of the linear ion trap | 200 | ms |
| Excitation time | 25 | ms |
| Entrance barrier Q3 | 8 | V |
| Inter-scan pause | 15 | ms |
| Trapping Q0 | Enabled | |
| Scan speed | 1000 | Da/sec |
| Curtain gas | 50.00 | psi |
| Cone voltage | 5500.00 | V |
| Source temperature | 550.00° | C. |
| Nebulizer gas (GS1) | 70.00 | psi |
| Heating gas (GS2) | 60.00 | psi |
| Collision gas | high | |
| Orifice potential (DP) | 90 | V |
| Collision energy (EC) | 31 | V |
| Collision energy spread (CES) | 0 | V |
| Excitation energy (AF2) | 0.11 | V |
| Input potential (IP) | 10.00 | V |
| Collision cell output potential (CXP) | 12.00 | V |

| | |
|---|---|
| Software version | Analyst 1.7.2 |

### Example 2. First Results

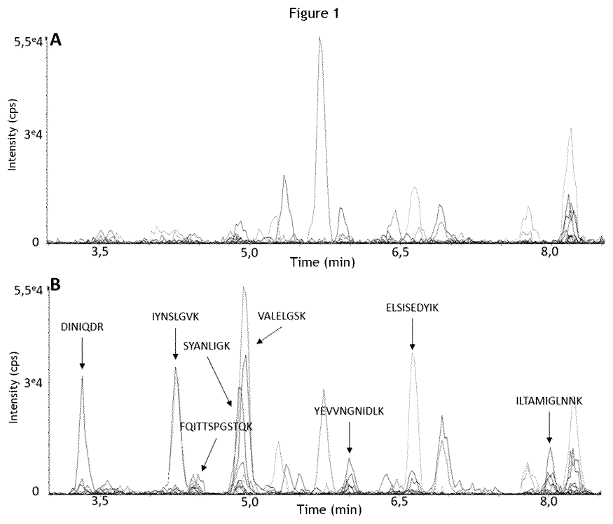

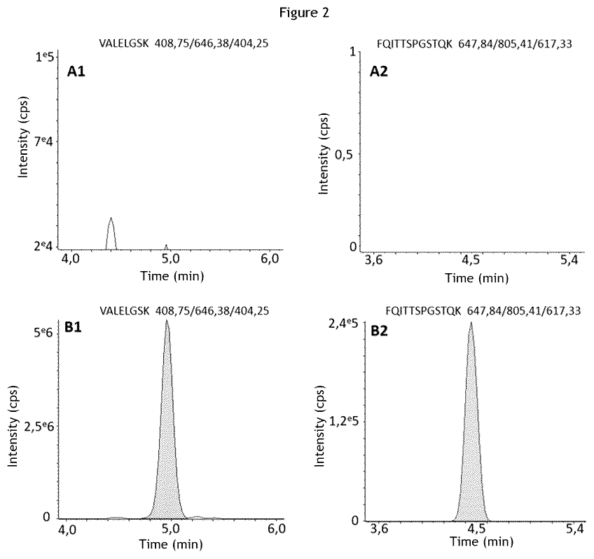

FIGS. 1 and 2 illustrate the necessity of the induction step for the detection of peptides derived from PBP2a by MRM.

Chromatograms A are derived from the analysis of strains without an induction step, chromatograms B correspond to the analysis of the same strains after an induction step.

In the absence of induction, the peaks of the peptides derived from PBP2a are confused with the background noise and therefore not detectable.

On the contrary, after an induction step, the characteristic peaks are observed: They are correctly drawn and above the background noise. PBP2a is correctly detected.

Blood cultures were inoculated for 98 strains of MRSA and 19 strains of methicillin-sensitive Staphylococcus aureus (MSSA) not expressing PBP2a, then the samples were prepared at bottle positivity and analysed according to the protocols of Example 1.

The area under the curve was measured for each transition. Although not expressing PBP2a, the area under the curve was also measured for MSSA-sensitive strain samples on the peptide elution window, to obtain a baseline value. Since the signal of a transition can be polluted by noise or interference due to the matrix, it is therefore necessary to evaluate this baseline in a matrix not containing the analyte (here PBP2a) in order to establish thresholds above which we can confirm the presence of peptides.

For each transition, the threshold corresponds to 150% of the maximum area measured over the peptide elution window for the MSSA control samples.

### Example 3. MRM Spectrometry Results

All MRM transitions for which the area value is greater than the threshold value were considered positive and are denoted as 1 in the following result tables. Transitions for which the area is less than the threshold value are denoted as 0 and therefore considered to be negative. A sample was considered to comprise an MRSA strain in cases where at least 3 peptides are detected with at least 2 positive transitions.

Tables 9 and 10 below correspond to the results obtained during the MRM analysis of the 98 MRSA without the induction step.

Tables 11 and 12 below correspond to the results obtained during the MRM analysis of the 98 MRSA with the induction step.

TABLE 9
Validation table of PBP2a MRM transitions from MRSA1b
to MRSA49b strains prepared WITHOUT induction step

| | Transition number | | | | | | | | | | | | | | | |
|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | 16 |
| MRSA1b | 1 | 0 | 1 | 1 | 1 | 1 | 1 | 1 | 1 | 1 | 1 | 1 | 1 | 1 | 1 | 1 |
| MRSA2b | 1 | 1 | 1 | 1 | 1 | 1 | 1 | 1 | 1 | 1 | 1 | 1 | 1 | 1 | 1 | 1 |
| MRSA3b | 0 | 0 | 0 | 0 | 0 | 0 | 0 | 0 | 0 | 0 | 0 | 0 | 0 | 0 | 0 | 0 |
| MRSA4b | 0 | 0 | 0 | 0 | 0 | 0 | 0 | 0 | 0 | 0 | 0 | 0 | 0 | 0 | 0 | 0 |
| MRSA5b | 00 | 0 | 0 | 0 | 0 | 0 | 0 | 0 | 0 | 0 | 1 | 0 | 0 | 0 | 0 | 0 |
| MRSA6b | 0 | 0 | 0 | 0 | 0 | 1 | 1 | 1 | 1 | 1 | 1 | 1 | 1 | 1 | 1 | 1 |
| MRSA7b | 0 | 0 | 0 | 0 | 0 | 0 | 0 | 0 | 1 | 0 | 1 | 0 | 0 | 0 | 0 | 0 |
| MRSA8b | 0 | 0 | 0 | 0 | 0 | 0 | 0 | 0 | 0 | 0 | 0 | 0 | 0 | 0 | 0 | 0 |
| MRSA9b | 0 | 0 | 0 | 0 | 0 | 0 | 0 | 0 | 0 | 0 | 0 | 0 | 0 | 0 | 0 | 0 |
| MRSA10b | 0 | 0 | 0 | 0 | 0 | 1 | 0 | 0 | 1 | 0 | 1 | 0 | 0 | 0 | 0 | 0 |
| MRSA11b | 0 | 0 | 0 | 0 | 0 | 0 | 0 | 0 | 1 | 1 | 1 | 0 | 1 | 1 | 0 | 0 |
| MRSA12b | 0 | 0 | 0 | 0 | 0 | 0 | 0 | 0 | 0 | 0 | 1 | 0 | 0 | 0 | 0 | 0 |
| MRSA13b | 0 | 0 | 0 | 0 | 0 | 0 | 0 | 0 | 0 | 0 | 0 | 0 | 0 | 0 | 0 | 0 |
| MRSA14b | 0 | 0 | 0 | 0 | 0 | 0 | 0 | 0 | 1 | 0 | 1 | 0 | 0 | 0 | 0 | 0 |
| MRSA15b | 0 | 0 | 0 | 0 | 0 | 0 | 0 | 0 | 1 | 1 | 1 | 1 | 0 | 1 | 0 | 0 |
| MRSA16b | 0 | 0 | 0 | 0 | 0 | 1 | 0 | 0 | 1 | 0 | 1 | 1 | 1 | 1 | 0 | 0 |
| MRSA17b | 0 | 0 | 0 | 0 | 0 | 0 | 0 | 0 | 1 | 0 | 1 | 0 | 1 | 0 | 0 | 0 |
| MRSA18b | 0 | 0 | 0 | 0 | 0 | 0 | 0 | 0 | 0 | 0 | 1 | 0 | 1 | 0 | 0 | 0 |
| MRSA19b | 0 | 0 | 0 | 0 | 0 | 0 | 0 | 0 | 0 | 0 | 1 | 0 | 0 | 0 | 0 | 0 |
| MRSA20b | 1 | 1 | 1 | 1 | 1 | 1 | 1 | 1 | 1 | 1 | 1 | 1 | 1 | 1 | 1 | 1 |
| MRSA21b | 0 | 0 | 0 | 0 | 0 | 0 | 1 | 1 | 1 | 1 | 1 | 0 | 1 | 1 | 0 | 1 |
| MRSA22b | 0 | 0 | 0 | 0 | 0 | 0 | 0 | 0 | 0 | 0 | 1 | 0 | 0 | 0 | 0 | 0 |
| MRSA23b | 0 | 0 | 0 | 0 | 0 | 0 | 0 | 0 | 1 | 0 | 1 | 0 | 0 | 1 | 0 | 0 |
| MRSA24b | 0 | 0 | 0 | 0 | 0 | 0 | 0 | 0 | 0 | 0 | 0 | 0 | 0 | 0 | 0 | 0 |
| MRSA25b | 0 | 0 | 0 | 0 | 0 | 1 | 0 | 0 | 1 | 1 | 1 | 0 | 1 | 0 | 0 | 0 |
| MRSA26b | 0 | 0 | 0 | 0 | 0 | 0 | 0 | 0 | 1 | 0 | 0 | 0 | 0 | 0 | 0 | 0 |
| MRSA27b | 0 | 0 | 0 | 0 | 0 | 0 | 0 | 0 | 0 | 0 | 0 | 0 | 0 | 0 | 0 | 0 |
| MRSA28b | 0 | 0 | 0 | 0 | 0 | 0 | 0 | 0 | 0 | 0 | 0 | 0 | 0 | 0 | 0 | 0 |
| MRSA29b | 0 | 0 | 0 | 0 | 0 | 0 | 0 | 0 | 0 | 0 | 1 | 0 | 0 | 1 | 0 | 0 |
| MRSA30b | 0 | 0 | 0 | 0 | 0 | 0 | 0 | 0 | 1 | 0 | 1 | 0 | 1 | 1 | 0 | 0 |
| MRSA31b | 0 | 0 | 0 | 0 | 0 | 1 | 1 | 0 | 0 | 0 | 1 | 0 | 0 | 1 | 0 | 0 |
| MRSA32b | 1 | 1 | 1 | 1 | 1 | 1 | 1 | 1 | 1 | 1 | 1 | 1 | 1 | 1 | 1 | 1 |
| MRSA33b | 1 | 1 | 1 | 1 | 1 | 1 | 1 | 1 | 1 | 1 | 1 | 1 | 1 | 1 | 1 | 1 |
| MRSA34b | 0 | 0 | 0 | 0 | 0 | 0 | 0 | 1 | 0 | 0 | 1 | 0 | 1 | 1 | 0 | 0 |
| MRSA35b | 0 | 0 | 0 | 0 | 0 | 0 | 0 | 0 | 1 | 0 | 1 | 0 | 1 | 0 | 0 | 0 |
| MRSA36b | 1 | 0 | 0 | 1 | 1 | 1 | 1 | 1 | 1 | 1 | 1 | 1 | 1 | 1 | 1 | 1 |
| MRSA37b | 0 | 0 | 0 | 0 | 0 | 0 | 0 | 0 | 0 | 0 | 0 | 0 | 0 | 0 | 0 | 0 |
| MRSA38b | 0 | 0 | 0 | 1 | 1 | 1 | 1 | 1 | 1 | 0 | 1 | 0 | 1 | 0 | 0 | 0 |
| MRSA39b | 0 | 0 | 0 | 1 | 1 | 1 | 1 | 1 | 0 | 1 | 1 | 0 | 0 | 1 | 0 | 0 |
| MRSA40b | 0 | 0 | 0 | 1 | 1 | 1 | 1 | 1 | 1 | 0 | 1 | 0 | 1 | 0 | 1 | 0 |
| MRSA41b | 1 | 0 | 0 | 1 | 1 | 1 | 1 | 1 | 1 | 0 | 1 | 0 | 1 | 1 | 1 | 1 |
| MRSA42b | 1 | 1 | 1 | 1 | 1 | 1 | 1 | 1 | 1 | 1 | 1 | 1 | 1 | 1 | 1 | 1 |
| MRSA43b | 1 | 1 | 1 | 1 | 1 | 1 | 1 | 1 | 1 | 1 | 1 | 1 | 1 | 1 | 1 | 1 |
| MRSA44b | 0 | 0 | 0 | 1 | 1 | 1 | 1 | 1 | 1 | 1 | 1 | 1 | 0 | 0 | 0 | 0 |
| MRSA45b | 1 | 0 | 0 | 1 | 1 | 1 | 1 | 1 | 1 | 1 | 1 | 1 | 1 | 1 | 1 | 0 |
| MRSA46b | 0 | 0 | 0 | 0 | 0 | 0 | 0 | 0 | 0 | 0 | 1 | 0 | 0 | 0 | 0 | 0 |
| MRSA47b | 0 | 0 | 0 | 0 | 0 | 0 | 0 | 0 | 0 | 0 | 1 | 0 | 0 | 1 | 0 | 0 |
| MRSA48b | 0 | 0 | 0 | 0 | 0 | 0 | 0 | 0 | 0 | 0 | 1 | 0 | 0 | 0 | 0 | 0 |
| MRSA49b | 0 | 0 | 0 | 0 | 0 | 0 | 0 | 0 | 0 | 0 | 0 | 0 | 0 | 0 | 0 | 0 |
| | Transition number | | | | | | | | | | | | | | | |
| | 17 | 18 | 19 | 20 | 21 | 22 | 23 | 24 | 25 | 26 | 27 | 28 | 29 | 30 | 31 | 32 |
| MRSA1b | 1 | 0 | 1 | 0 | 0 | 0 | 1 | 0 | 0 | 0 | 0 | 0 | 1 | 1 | 1 | 1 |
| MRSA2b | 1 | 1 | 1 | 1 | 0 | 1 | 1 | 1 | 1 | 1 | 1 | 1 | 1 | 1 | 1 | 1 |
| MRSA3b | 0 | 0 | 0 | 0 | 0 | 0 | 0 | 0 | 0 | 0 | 0 | 0 | 0 | 0 | 0 | 0 |
| MRSA4b | 0 | 0 | 0 | 0 | 0 | 0 | 0 | 0 | 0 | 0 | 0 | 0 | 0 | 0 | 0 | 0 |
| MRSA5b | 0 | 0 | 0 | 0 | 0 | 0 | 0 | 0 | 0 | 0 | 0 | 0 | 0 | 0 | 0 | 0 |
| MRSA6b | 1 | 1 | 1 | 1 | 0 | 0 | 1 | 1 | 1 | 1 | 0 | 0 | 1 | 1 | 1 | 1 |
| MRSA7b | 0 | 0 | 0 | 0 | 0 | 0 | 0 | 0 | 0 | 0 | 0 | 0 | 0 | 0 | 0 | 0 |
| MRSA8b | 0 | 0 | 0 | 0 | 0 | 0 | 0 | 0 | 0 | 0 | 0 | 0 | 0 | 0 | 0 | 0 |
| MRSA9b | 0 | 0 | 0 | 0 | 0 | 0 | 0 | 0 | 0 | 0 | 0 | 0 | 0 | 0 | 0 | 0 |
| MRSA10b | 1 | 0 | 0 | 0 | 0 | 0 | 1 | 0 | 0 | 0 | 0 | 0 | 0 | 0 | 0 | 0 |
| MRSA11b | 0 | 0 | 1 | 0 | 0 | 0 | 1 | 0 | 1 | 1 | 1 | 1 | 1 | 0 | 1 | 0 |
| MRSA12b | 0 | 0 | 0 | 0 | 0 | 0 | 0 | 0 | 0 | 0 | 0 | 0 | 0 | 0 | 0 | 0 |
| MRSA13b | 0 | 0 | 0 | 0 | 0 | 0 | 0 | 0 | 0 | 0 | 0 | 0 | 0 | 0 | 0 | 0 |
| MRSA14b | 0 | 0 | 1 | 0 | 0 | 0 | 1 | 0 | 0 | 0 | 0 | 0 | 0 | 0 | 0 | 0 |
| MRSA15b | 0 | 0 | 1 | 0 | 0 | 0 | 1 | 0 | 0 | 0 | 0 | 0 | 0 | 0 | 0 | 0 |
| MRSA16b | 0 | 0 | 1 | 0 | 0 | 0 | 1 | 0 | 0 | 0 | 0 | 0 | 0 | 1 | 1 | 0 |
| MRSA17b | 0 | 0 | 1 | 0 | 0 | 0 | 0 | 0 | 0 | 0 | 0 | 0 | 0 | 1 | 0 | 0 |
| MRSA18b | 0 | 0 | 1 | 0 | 0 | 0 | 1 | 0 | 0 | 0 | 0 | 0 | 0 | 0 | 0 | 0 |
| MRSA19b | 0 | 0 | 0 | 0 | 0 | 0 | 0 | 0 | 0 | 0 | 0 | 0 | 0 | 0 | 0 | 0 |
| MRSA20b | 1 | 1 | 1 | 0 | 0 | 0 | 1 | 0 | 0 | 0 | 0 | 1 | 1 | 1 | 1 | 1 |
| MRSA21b | 0 | 0 | 1 | 0 | 0 | 0 | 1 | 0 | 0 | 0 | 0 | 0 | 1 | 0 | 0 | 0 |
| MRSA22b | 0 | 0 | 0 | 0 | 0 | 0 | 0 | 0 | 1 | 1 | 1 | 0 | 0 | 0 | 0 | 0 |
| MRSA23b | 0 | 0 | 1 | 0 | 0 | 0 | 0 | 0 | 0 | 0 | 0 | 0 | 0 | 0 | 0 | 0 |
| MRSA24b | 0 | 0 | 0 | 0 | 0 | 0 | 0 | 0 | 0 | 0 | 0 | 0 | 0 | 0 | 0 | 0 |
| MRSA25b | 0 | 0 | 1 | 0 | 0 | 0 | 1 | 0 | 0 | 0 | 0 | 0 | 0 | 0 | 1 | 0 |
| MRSA26b | 0 | 0 | 0 | 0 | 0 | 0 | 0 | 0 | 0 | 0 | 0 | 0 | 0 | 0 | 0 | 0 |
| MRSA27b | 0 | 0 | 0 | 0 | 0 | 0 | 0 | 0 | 0 | 0 | 0 | 0 | 0 | 0 | 0 | 0 |
| MRSA28b | 0 | 0 | 0 | 0 | 0 | 0 | 0 | 0 | 0 | 0 | 0 | 0 | 0 | 0 | 0 | 0 |
| MRSA29b | 0 | 0 | 0 | 0 | 0 | 0 | 0 | 0 | 0 | 0 | 0 | 0 | 0 | 0 | 0 | 0 |
| MRSA30b | 0 | 0 | 1 | 0 | 0 | 0 | 1 | 0 | 0 | 0 | 0 | 0 | 1 | 0 | 1 | 0 |
| MRSA31b | 0 | 0 | 1 | 0 | 0 | 0 | 0 | 0 | 0 | 0 | 0 | 0 | 1 | 0 | 0 | 0 |
| MRSA32b | 1 | 0 | 1 | 0 | 0 | 0 | 1 | 0 | 0 | 0 | 0 | 0 | 1 | 1 | 1 | 1 |
| MRSA33b | 1 | 0 | 01 | 0 | 0 | 0 | 1 | 0 | 1 | 1 | 1 | 1 | 1 | 1 | 1 | 1 |
| MRSA34b | 0 | 0 | 1 | 0 | 0 | 0 | 0 | 0 | 0 | 0 | 0 | 0 | 1 | 0 | 0 | 0 |
| MRSA35b | 0 | 0 | 0 | 0 | 0 | 0 | 0 | 0 | 0 | 0 | 0 | 0 | 0 | 0 | 0 | 0 |
| MRSA36b | 1 | 0 | 1 | 0 | 0 | 0 | 1 | 0 | 0 | 0 | 0 | 0 | 1 | 1 | 1 | 0 |
| MRSA37b | 0 | 0 | 0 | 0 | 0 | 0 | 0 | 0 | 0 | 0 | 0 | 0 | 0 | 0 | 0 | 0 |
| MRSA38b | 0 | 0 | 1 | 0 | 0 | 0 | 1 | 0 | 0 | 0 | 0 | 0 | 1 | 0 | 1 | 0 |
| MRSA39b | 1 | 0 | 1 | 0 | 0 | 0 | 1 | 0 | 0 | 0 | 0 | 0 | 0 | 0 | 1 | 0 |
| MRSA40b | 1 | 0 | 1 | 0 | 0 | 0 | 1 | 0 | 0 | 0 | 0 | 0 | 0 | 0 | 1 | 0 |
| MRSA41b | 1 | 0 | 1 | 0 | 0 | 0 | 1 | 0 | 1 | 1 | 1 | 1 | 1 | 1 | 1 | 0 |
| MRSA42b | 1 | 0 | 1 | 0 | 0 | 0 | 1 | 0 | 1 | 1 | 0 | 1 | 1 | 1 | 1 | 1 |
| MRSA43b | 1 | 1 | 1 | 0 | 0 | 0 | 1 | 1 | 0 | 0 | 0 | 0 | 1 | 1 | 1 | 1 |
| MRSA44b | 1 | 0 | 1 | 0 | 0 | 0 | 1 | 0 | 0 | 0 | 0 | 0 | 0 | 0 | 1 | 1 |
| MRSA45b | 0 | 0 | 1 | 0 | 0 | 0 | 1 | 0 | 0 | 0 | 0 | 0 | 0 | 0 | 1 | 0 |
| MRSA46b | 0 | 0 | 0 | 0 | 0 | 0 | 0 | 0 | 0 | 0 | 0 | 0 | 0 | 0 | 0 | 0 |
| MRSA47b | 0 | 0 | 1 | 0 | 0 | 0 | 0 | 0 | 0 | 0 | 0 | 0 | 0 | 0 | 0 | 0 |
| MRSA48b | 0 | 0 | 0 | 0 | 0 | 0 | 0 | 0 | 0 | 0 | 0 | 0 | 0 | 0 | 0 | 0 |
| MRSA49b | 0 | 0 | 0 | 0 | 0 | 0 | 0 | 0 | 0 | 0 | 0 | 0 | 0 | 0 | 0 | 0 |

TABLE 10
Validation table of PBP2a MRM transitions from MRSA50b
to MRSA98b strains prepared WITHOUT induction step

| | Transition number | | | | | | | | | | | | | | | |
|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | 16 |
| MRSA50b | 0 | 0 | 0 | 0 | 0 | 0 | 0 | 0 | 0 | 0 | 0 | 0 | 0 | 0 | 0 | 0 |
| MRSA51b | 0 | 0 | 0 | 0 | 0 | 0 | 0 | 0 | 0 | 0 | 1 | 0 | 0 | 0 | 0 | 0 |
| MRSA52b | 0 | 0 | 0 | 0 | 0 | 0 | 0 | 0 | 0 | 0 | 0 | 0 | 0 | 0 | 0 | 0 |
| MRSA53b | 0 | 0 | 0 | 0 | 0 | 0 | 0 | 0 | 1 | 0 | 1 | 0 | 0 | 0 | 0 | 0 |
| MRSA54b | 0 | 0 | 0 | 0 | 0 | 0 | 1 | 0 | 1 | 0 | 1 | 0 | 1 | 1 | 0 | 0 |
| MRSA55b | 0 | 0 | 0 | 0 | 0 | 1 | 1 | 0 | 0 | 0 | 1 | 0 | 0 | 0 | 0 | 0 |
| MRSA56b | 0 | 0 | 0 | 0 | 0 | 0 | 0 | 0 | 0 | 0 | 1 | 0 | 0 | 0 | 0 | 0 |
| MRSA57b | 0 | 0 | 0 | 0 | 0 | 1 | 0 | 0 | 1 | 0 | 1 | 1 | 1 | 1 | 0 | 0 |
| MRSA58b | 1 | 0 | 0 | 0 | 0 | 1 | 1 | 1 | 1 | 0 | 1 | 1 | 1 | 1 | 1 | 0 |
| MRSA59b | 1 | 0 | 0 | 1 | 1 | 1 | 1 | 1 | 1 | 1 | 1 | 1 | 1 | 1 | 1 | 1 |
| MRSA60b | 0 | 0 | 0 | 0 | 0 | 1 | 1 | 0 | 1 | 0 | 1 | 0 | 1 | 0 | 0 | 0 |
| MRSA61b | 0 | 0 | 0 | 0 | 0 | 0 | 0 | 0 | 0 | 0 | 1 | 0 | 0 | 0 | 0 | 0 |
| MRSA62b | 1 | 1 | 1 | 1 | 1 | 1 | 1 | 1 | 1 | 1 | 1 | 1 | 1 | 1 | 1 | 1 |
| MRSA63b | 0 | 0 | 0 | 0 | 0 | 0 | 0 | 0 | 0 | 0 | 1 | 0 | 1 | 1 | 0 | 0 |
| MRSA64b | 0 | 0 | 0 | 0 | 0 | 0 | 0 | 1 | 0 | 0 | 1 | 0 | 0 | 0 | 0 | 0 |
| MRSA65b | 0 | 0 | 0 | 0 | 0 | 0 | 1 | 0 | 1 | 0 | 1 | 1 | 1 | 1 | 0 | 0 |
| MRSA66b | 0 | 0 | 0 | 0 | 0 | 0 | 1 | 0 | 1 | 0 | 1 | 0 | 1 | 1 | 0 | 1 |
| MRSA67b | 0 | 0 | 0 | 0 | 0 | 0 | 0 | 0 | 0 | 0 | 1 | 0 | 0 | 1 | 0 | 0 |
| MRSA68b | 1 | 1 | 1 | 0 | 0 | 0 | 0 | 0 | 1 | 0 | 1 | 1 | 0 | 1 | 0 | 0 |
| MRSA69b | 0 | 0 | 0 | 0 | 0 | 1 | 0 | 0 | 0 | 0 | 1 | 0 | 1 | 0 | 0 | 0 |
| MRSA70b | 0 | 0 | 0 | 0 | 0 | 0 | 0 | 0 | 1 | 0 | 1 | 0 | 1 | 0 | 0 | 0 |
| MRSA71b | 1 | 1 | 1 | 0 | 0 | 0 | 0 | 0 | 1 | 1 | 1 | 1 | 1 | 1 | 1 | 1 |
| MRSA72b | 1 | 1 | 1 | 0 | 0 | 0 | 0 | 0 | 1 | 1 | 1 | 1 | 1 | 1 | 1 | 1 |
| MRSA73b | 0 | 0 | 0 | 0 | 0 | 0 | 0 | 0 | 0 | 0 | 0 | 0 | 0 | 0 | 0 | 0 |
| MRSA74b | 1 | 0 | 1 | 0 | 0 | 0 | 0 | 0 | 1 | 1 | 1 | 1 | 1 | 1 | 1 | 1 |
| MRSA75b | 0 | 0 | 0 | 0 | 0 | 0 | 0 | 0 | 0 | 0 | 0 | 0 | 0 | 0 | 0 | 0 |
| MRSA76b | 1 | 1 | 1 | 0 | 0 | 0 | 0 | 0 | 1 | 1 | 1 | 1 | 1 | 1 | 1 | 1 |
| MRSA77b | 0 | 0 | 0 | 0 | 0 | 0 | 0 | 0 | 1 | 0 | 1 | 0 | 0 | 0 | 0 | 0 |
| MRSA78b | 1 | 0 | 1 | 0 | 0 | 0 | 0 | 0 | 1 | 1 | 1 | 1 | 1 | 1 | 1 | 1 |
| MRSA79b | 0 | 0 | 0 | 0 | 0 | 0 | 0 | 0 | 1 | 1 | 1 | 0 | 1 | 1 | 0 | 0 |
| MRSA80b | 1 | 0 | 1 | 0 | 0 | 0 | 0 | 0 | 1 | 1 | 1 | 1 | 1 | 1 | 1 | 1 |
| MRSA81b | 0 | 0 | 0 | 0 | 0 | 0 | 0 | 0 | 1 | 0 | 1 | 0 | 0 | 1 | 0 | 0 |
| MRSA82b | 1 | 0 | 0 | 0 | 0 | 0 | 0 | 0 | 1 | 0 | 1 | 0 | 1 | 0 | 0 | 1 |
| MRSA83b | 0 | 0 | 0 | 0 | 0 | 0 | 0 | 0 | 1 | 0 | 1 | 0 | 0 | 1 | 0 | 0 |
| MRSA84b | 0 | 0 | 0 | 0 | 0 | 0 | 0 | 0 | 0 | 0 | 1 | 0 | 0 | 0 | 0 | 0 |
| MRSA85b | 0 | 0 | 0 | 0 | 0 | 0 | 0 | 0 | 0 | 0 | 1 | 0 | 0 | 1 | 0 | 0 |
| MRSA86b | 1 | 0 | 1 | 0 | 0 | 0 | 0 | 0 | 1 | 1 | 1 | 0 | 1 | 1 | 1 | 1 |
| MRSA87b | 0 | 0 | 0 | 0 | 0 | 0 | 0 | 0 | 1 | 0 | 1 | 0 | 1 | 0 | 0 | 0 |
| MRSA88b | 0 | 0 | 0 | 0 | 0 | 0 | 0 | 0 | 0 | 0 | 1 | 0 | 0 | 0 | 0 | 0 |
| MRSA89b | 0 | 0 | 0 | 0 | 0 | 0 | 0 | 0 | 1 | 0 | 1 | 0 | 0 | 0 | 0 | 0 |
| MRSA90b | 0 | 0 | 0 | 0 | 0 | 0 | 0 | 0 | 0 | 0 | 1 | 0 | 1 | 0 | 0 | 0 |
| MRSA91b | 1 | 1 | 1 | 1 | 1 | 1 | 1 | 1 | 1 | 1 | 1 | 1 | 1 | 1 | 1 | 1 |
| MRSA92b | 1 | 0 | 1 | 0 | 0 | 0 | 0 | 0 | 1 | 1 | 1 | 1 | 1 | 1 | 1 | 1 |
| MRSA93b | 0 | 0 | 0 | 0 | 0 | 0 | 0 | 0 | 0 | 0 | 0 | 0 | 0 | 0 | 0 | 0 |
| MRSA94b | 1 | 0 | 0 | 0 | 0 | 0 | 0 | 0 | 1 | 1 | 1 | 1 | 1 | 1 | 1 | 1 |
| MRSA95b | 0 | 0 | 0 | 1 | 1 | 1 | 1 | 1 | 1 | 1 | 1 | 1 | 1 | 1 | 1 | 1 |
| MRSA96b | 0 | 0 | 0 | 0 | 0 | 0 | 0 | 0 | 1 | 1 | 1 | 0 | 0 | 1 | 0 | 0 |
| MRSA97b | 0 | 0 | 0 | 0 | 0 | 0 | 0 | 0 | 1 | 1 | 1 | 1 | 0 | 0 | 0 | 0 |
| MRSA98b | 0 | 0 | 0 | 0 | 0 | 0 | 0 | 0 | 1 | 1 | 1 | 0 | 1 | 1 | 0 | 0 |
| | Transition number | | | | | | | | | | | | | | | |
| | 17 | 18 | 19 | 20 | 21 | 22 | 23 | 24 | 25 | 26 | 27 | 28 | 29 | 30 | 31 | 32 |
| MRSA50b | 0 | 0 | 0 | 0 | 0 | 0 | 0 | 0 | 0 | 0 | 0 | 0 | 0 | 0 | 0 | 0 |
| MRSA51b | 0 | 0 | 1 | 0 | 0 | 0 | 0 | 0 | 0 | 0 | 0 | 0 | 0 | 0 | 0 | 0 |
| MRSA52b | 0 | 0 | 0 | 0 | 0 | 0 | 0 | 0 | 0 | 0 | 0 | 0 | 0 | 0 | 0 | 0 |
| MRSA53b | 0 | 0 | 0 | 0 | 0 | 0 | 0 | 0 | 0 | 0 | 0 | 0 | 1 | 0 | 0 | 0 |
| MRSA54b | 1 | 0 | 1 | 0 | 0 | 0 | 1 | 0 | 0 | 0 | 0 | 0 | 1 | 0 | 1 | 0 |
| MRSA55b | 0 | 0 | 0 | 0 | 0 | 0 | 0 | 0 | 0 | 0 | 0 | 0 | 0 | 0 | 0 | 0 |
| MRSA56b | 0 | 0 | 0 | 0 | 0 | 0 | 0 | 0 | 0 | 0 | 0 | 0 | 0 | 0 | 0 | 0 |
| MRSA57b | 0 | 0 | 1 | 0 | 0 | 0 | 1 | 0 | 0 | 0 | 0 | 0 | 1 | 0 | 0 | 0 |
| MRSA58b | 0 | 0 | 1 | 0 | 0 | 0 | 1 | 0 | 1 | 0 | 0 | 0 | 1 | 1 | 1 | 1 |
| MRSA59b | 1 | 0 | 1 | 0 | 0 | 0 | 1 | 0 | 0 | 0 | 0 | 0 | 1 | 1 | 1 | 1 |
| MRSA60b | 0 | 0 | 1 | 0 | 0 | 0 | 0 | 0 | 1 | 1 | 1 | 1 | 1 | 0 | 0 | 0 |
| MRSA61b | 0 | 0 | 1 | 0 | 0 | 0 | 0 | 0 | 0 | 0 | 0 | 0 | 0 | 0 | 0 | 0 |
| MRSA62b | 1 | 1 | 1 | 1 | 1 | 0 | 1 | 1 | 0 | 0 | 0 | 0 | 1 | 1 | 1 | 1 |
| MRSA63b | 0 | 0 | 1 | 0 | 0 | 0 | 1 | 0 | 0 | 0 | 0 | 0 | 1 | 1 | 1 | 0 |
| MRSA64b | 0 | 0 | 1 | 0 | 0 | 0 | 1 | 0 | 0 | 0 | 0 | 0 | 0 | 0 | 0 | 0 |
| MRSA65b | 0 | 0 | 1 | 0 | 0 | 0 | 1 | 0 | 0 | 0 | 0 | 0 | 1 | 1 | 1 | 0 |
| MRSA66b | 0 | 0 | 1 | 0 | 0 | 0 | 1 | 0 | 0 | 0 | 0 | 0 | 1 | 1 | 0 | 0 |
| MRSA67b | 0 | 0 | 1 | 0 | 0 | 0 | 1 | 0 | 0 | 0 | 0 | 0 | 0 | 0 | 0 | 0 |
| MRSA68b | 1 | 0 | 1 | 0 | 0 | 0 | 1 | 0 | 0 | 0 | 0 | 0 | 1 | 0 | 0 | 0 |
| MRSA69b | 0 | 0 | 1 | 0 | 0 | 0 | 0 | 0 | 1 | 1 | 0 | 1 | 0 | 0 | 0 | 0 |
| MRSA70b | 0 | 0 | 1 | 0 | 0 | 0 | 1 | 0 | 1 | 1 | 0 | 1 | 0 | 0 | 0 | 0 |
| MRSA71b | 1 | 0 | 1 | 0 | 0 | 0 | 1 | 0 | 0 | 0 | 0 | 0 | 1 | 1 | 1 | 1 |
| MRSA72b | 1 | 0 | 1 | 0 | 0 | 0 | 1 | 1 | 1 | 1 | 0 | 1 | 1 | 1 | 1 | 1 |
| MRSA73b | 0 | 0 | 0 | 0 | 0 | 0 | 0 | 0 | 0 | 0 | 0 | 0 | 0 | 0 | 0 | 0 |
| MRSA74b | 1 | 0 | 1 | 0 | 0 | 0 | 1 | 0 | 1 | 1 | 1 | 1 | 1 | 1 | 1 | 1 |
| MRSA75b | 0 | 0 | 0 | 0 | 0 | 0 | 0 | 0 | 0 | 0 | 0 | 0 | 0 | 0 | 0 | 0 |
| MRSA76b | 1 | 1 | 1 | 0 | 0 | 0 | 1 | 0 | 1 | 1 | 0 | 1 | 1 | 1 | 1 | 1 |
| MRSA77b | 0 | 0 | 0 | 0 | 0 | 0 | 1 | 0 | 0 | 0 | 0 | 0 | 0 | 0 | 0 | 0 |
| MRSA78b | 1 | 0 | 1 | 0 | 0 | 0 | 1 | 0 | 1 | 1 | 1 | 1 | 1 | 1 | 1 | 1 |
| MRSA79b | 1 | 0 | 0 | 0 | 0 | 0 | 1 | 0 | 0 | 0 | 0 | 0 | 0 | 0 | 0 | 0 |
| MRSA80b | 1 | 0 | 1 | 0 | 0 | 0 | 1 | 0 | 0 | 0 | 0 | 0 | 1 | 1 | 1 | 1 |
| MRSA81b | 0 | 0 | 1 | 0 | 0 | 0 | 1 | 0 | 0 | 0 | 0 | 0 | 1 | 0 | 0 | 0 |
| MRSA82b | 0 | 0 | 1 | 0 | 0 | 0 | 1 | 0 | 0 | 0 | 0 | 0 | 0 | 0 | 0 | 0 |
| MRSA83b | 0 | 0 | 1 | 0 | 0 | 0 | 1 | 0 | 0 | 0 | 0 | 0 | 1 | 0 | 1 | 0 |
| MRSA84b | 0 | 0 | 0 | 0 | 0 | 0 | 0 | 0 | 1 | 1 | 0 | 0 | 0 | 0 | 0 | 0 |
| MRSA85b | 0 | 0 | 0 | 0 | 0 | 0 | 1 | 0 | 0 | 0 | 0 | 0 | 0 | 0 | 0 | 0 |
| MRSA86b | 1 | 0 | 1 | 0 | 0 | 0 | 1 | 0 | 0 | 0 | 0 | 0 | 1 | 0 | 1 | 1 |
| MRSA87b | 0 | 0 | 0 | 0 | 0 | 0 | 0 | 0 | 0 | 0 | 0 | 0 | 1 | 0 | 0 | 0 |
| MRSA88b | 0 | 0 | 0 | 1 | 0 | 0 | 0 | 0 | 0 | 0 | 0 | 0 | 0 | 0 | 0 | 0 |
| MRSA89b | 0 | 0 | 1 | 0 | 0 | 0 | 1 | 0 | 1 | 1 | 1 | 1 | 0 | 0 | 0 | 0 |
| MRSA90b | 0 | 0 | 0 | 0 | 0 | 0 | 1 | 0 | 1 | 0 | 0 | 1 | 0 | 0 | 0 | 0 |
| MRSA91b | 1 | 1 | 1 | 1 | 0 | 0 | 1 | 1 | 0 | 0 | 0 | 0 | 1 | 1 | 1 | 1 |
| MRSA92b | 1 | 0 | 1 | 0 | 0 | 0 | 1 | 0 | 0 | 0 | 0 | 1 | 1 | 1 | 1 | 0 |
| MRSA93b | 0 | 0 | 0 | 0 | 0 | 0 | 0 | 0 | 0 | 0 | 0 | 0 | 0 | 0 | 0 | 0 |
| MRSA94b | 1 | 0 | 1 | 0 | 0 | 0 | 1 | 0 | 0 | 0 | 0 | 0 | 1 | 1 | 1 | 1 |
| MRSA95b | 1 | 0 | 1 | 0 | 0 | 0 | 1 | 0 | 1 | 1 | 0 | 0 | 1 | 1 | 1 | 1 |
| MRSA96b | 1 | 0 | 1 | 0 | 0 | 0 | 1 | 0 | 0 | 0 | 0 | 0 | 1 | 0 | 0 | 0 |
| MRSA97b | 0 | 0 | 0 | 0 | 0 | 0 | 1 | 0 | 0 | 0 | 0 | 0 | 1 | 0 | 0 | 0 |
| MRSA98b | 0 | 0 | 0 | 0 | 0 | 0 | 1 | 0 | 0 | 0 | 0 | 0 | 0 | 0 | 0 | 0 |

TABLE 11
Validation table of PBP2a MRM transitions from MRSA1b
to MRSA49b strains prepared WITH induction step

| | Transition number | | | | | | | | | | | | | | | |
|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | 16 |
| MRSA1b | 1 | 1 | 1 | 1 | 1 | 1 | 1 | 1 | 1 | 1 | 1 | 1 | 1 | 1 | 1 | 1 |
| MRSA2b | 1 | 1 | 1 | 1 | 1 | 1 | 1 | 1 | 1 | 1 | 1 | 1 | 1 | 1 | 1 | 1 |
| MRSA3b | 1 | 1 | 1 | 1 | 1 | 1 | 1 | 1 | 1 | 1 | 1 | 1 | 1 | 1 | 1 | 1 |
| MRSA4b | 1 | 0 | 1 | 1 | 1 | 1 | 1 | 1 | 1 | 1 | 1 | 1 | 1 | 1 | 1 | 1 |
| MRSA5b | 1 | 1 | 1 | 1 | 1 | 1 | 1 | 1 | 1 | 1 | 1 | 1 | 1 | 1 | 1 | 1 |
| MRSA6b | 1 | 1 | 1 | 1 | 1 | 1 | 1 | 1 | 1 | 1 | 1 | 1 | 1 | 1 | 1 | 1 |
| MRSA7b | 1 | 0 | 0 | 1 | 1 | 1 | 1 | 1 | 1 | 1 | 1 | 1 | 1 | 1 | 1 | 1 |
| MRSA8b | 1 | 0 | 0 | 1 | 1 | 1 | 1 | 1 | 1 | 1 | 1 | 1 | 1 | 1 | 1 | 1 |
| MRSA9b | 0 | 0 | 0 | 0 | 0 | 0 | 0 | 0 | 1 | 0 | 1 | 0 | 0 | 1 | 0 | 0 |
| MRSA10b | 0 | 0 | 0 | 1 | 1 | 1 | 1 | 1 | 1 | 1 | 1 | 1 | 1 | 1 | 1 | 1 |
| MRSA11b | 0 | 0 | 0 | 1 | 1 | 1 | 1 | 1 | 1 | 1 | 1 | 1 | 1 | 1 | 1 | 1 |
| MRSA12b | 1 | 0 | 1 | 1 | 0 | 1 | 1 | 1 | 1 | 1 | 1 | 1 | 1 | 1 | 1 | 1 |
| MRSA13b | 1 | 0 | 0 | 1 | 0 | 0 | 1 | 1 | 1 | 0 | 1 | 0 | 1 | 1 | 1 | 0 |
| MRSA14b | 1 | 0 | 0 | 1 | 0 | 1 | 1 | 0 | 1 | 1 | 1 | 0 | 1 | 1 | 0 | 1 |
| MRSA15b | 1 | 1 | 1 | 1 | 1 | 1 | 1 | 1 | 1 | 1 | 1 | 1 | 1 | 1 | 1 | 1 |
| MRSA16b | 1 | 1 | 1 | 1 | 1 | 1 | 1 | 1 | 1 | 1 | 1 | 1 | 1 | 1 | 1 | 1 |
| MRSA17b | 1 | 0 | 0 | 0 | 0 | 1 | 1 | 0 | 1 | 1 | 1 | 1 | 1 | 1 | 0 | 0 |
| MRSA18b | 1 | 1 | 1 | 1 | 1 | 1 | 1 | 1 | 1 | 1 | 1 | 1 | 1 | 1 | 1 | 1 |
| MRSA19b | 1 | 1 | 1 | 1 | 1 | 1 | 1 | 1 | 1 | 1 | 1 | 1 | 1 | 1 | 1 | 1 |
| MRSA20b | 1 | 1 | 1 | 1 | 1 | 1 | 1 | 1 | 1 | 1 | 1 | 1 | 1 | 1 | 1 | 1 |
| MRSA21b | 0 | 0 | 0 | 0 | 0 | 1 | 1 | 0 | 1 | 0 | 1 | 1 | 1 | 1 | 0 | 1 |
| MRSA22b | 1 | 1 | 1 | 1 | 1 | 1 | 1 | 1 | 1 | 1 | 1 | 1 | 1 | 1 | 1 | 1 |
| MRSA23b | 1 | 1 | 0 | 1 | 1 | 1 | 1 | 0 | 1 | 0 | 1 | 0 | 1 | 1 | 0 | 1 |
| MRSA24b | 0 | 0 | 0 | 0 | 0 | 1 | 1 | 0 | 1 | 0 | 1 | 0 | 0 | 1 | 0 | 0 |
| MRSA25b | 1 | 0 | 1 | 1 | 1 | 1 | 1 | 1 | 1 | 1 | 1 | 1 | 1 | 1 | 1 | 1 |
| MRSA26b | 1 | 1 | 1 | 1 | 1 | 1 | 1 | 1 | 1 | 1 | 1 | 1 | 1 | 1 | 1 | 1 |
| MRSA27b | 1 | 1 | 1 | 1 | 1 | 1 | 1 | 1 | 1 | 1 | 1 | 1 | 1 | 1 | 1 | 1 |
| MRSA28b | 1 | 1 | 1 | 1 | 1 | 1 | 1 | 1 | 1 | 1 | 1 | 1 | 1 | 1 | 1 | 1 |
| MRSA29b | 1 | 1 | 1 | 1 | 1 | 1 | 1 | 1 | 1 | 1 | 1 | 1 | 1 | 1 | 1 | 1 |
| MRSA30b | 1 | 1 | 1 | 1 | 1 | 1 | 1 | 1 | 1 | 1 | 1 | 1 | 1 | 1 | 1 | 1 |
| MRSA31b | 1 | 0 | 0 | 1 | 0 | 0 | 0 | 1 | 1 | 0 | 1 | 1 | 1 | 1 | 0 | 1 |
| MRSA32b | 1 | 1 | 1 | 1 | 1 | 1 | 1 | 1 | 1 | 1 | 1 | 1 | 1 | 1 | 1 | 1 |
| MRSA33b | 1 | 1 | 1 | 1 | 1 | 1 | 1 | 1 | 1 | 1 | 1 | 1 | 1 | 1 | 1 | 1 |
| MRSA34b | 1 | 1 | 1 | 1 | 1 | 1 | 1 | 1 | 1 | 1 | 1 | 1 | 1 | 1 | 1 | 1 |
| MRSA35b | 1 | 1 | 0 | 0 | 0 | 0 | 1 | 0 | 1 | 0 | 1 | 0 | 1 | 1 | 1 | 1 |
| MRSA36b | 1 | 1 | 1 | 1 | 1 | 1 | 1 | 1 | 1 | 1 | 1 | 1 | 1 | 1 | 1 | 1 |
| MRSA37b | 1 | 0 | 1 | 1 | 0 | 1 | 1 | 1 | 1 | 1 | 1 | 1 | 1 | 1 | 0 | 1 |
| MRSA38b | 1 | 1 | 1 | 1 | 1 | 1 | 1 | 1 | 1 | 1 | 1 | 1 | 1 | 1 | 1 | 1 |
| MRSA39b | 1 | 1 | 1 | 1 | 1 | 1 | 1 | 1 | 1 | 1 | 1 | 1 | 1 | 1 | 1 | 1 |
| MRSA40b | 1 | 1 | 1 | 1 | 1 | 1 | 1 | 1 | 1 | 1 | 1 | 1 | 1 | 1 | 1 | 1 |
| MRSA41b | 0 | 0 | 0 | 1 | 0 | 1 | 1 | 1 | 1 | 0 | 1 | 0 | 0 | 0 | 0 | 0 |
| MRSA42b | 1 | 1 | 1 | 1 | 1 | 1 | 1 | 1 | 1 | 1 | 1 | 1 | 1 | 1 | 1 | 1 |
| MRSA43b | 1 | 1 | 1 | 1 | 1 | 1 | 1 | 1 | 1 | 1 | 1 | 1 | 1 | 1 | 1 | 1 |
| MRSA44b | 1 | 1 | 1 | 1 | 1 | 1 | 1 | 1 | 1 | 1 | 1 | 1 | 1 | 1 | 1 | 1 |
| MRSA45b | 1 | 1 | 1 | 1 | 1 | 1 | 1 | 1 | 1 | 1 | 1 | 1 | 1 | 1 | 1 | 1 |
| MRSA46b | 1 | 1 | 1 | 1 | 1 | 1 | 1 | 1 | 1 | 1 | 1 | 1 | 1 | 1 | 1 | 1 |
| MRSA47b | 1 | 0 | 0 | 1 | 0 | 1 | 1 | 1 | 1 | 0 | 1 | 1 | 1 | 1 | 0 | 0 |
| MRSA48b | 1 | 0 | 0 | 1 | 1 | 1 | 1 | 1 | 1 | 1 | 1 | 1 | 1 | 1 | 0 | 1 |
| MRSA49b | 1 | 1 | 0 | 1 | 1 | 1 | 1 | 1 | 1 | 1 | 1 | 1 | 1 | 1 | 1 | 1 |
| | Transition number | | | | | | | | | | | | | | | |
| | 17 | 18 | 19 | 20 | 21 | 22 | 23 | 24 | 25 | 26 | 27 | 28 | 29 | 30 | 31 | 32 |
| MRSA1b | 1 | 1 | 1 | 1 | 0 | 0 | 1 | 0 | 1 | 1 | 1 | 1 | 1 | 1 | 1 | 1 |
| MRSA2b | 1 | 1 | 1 | 1 | 1 | 0 | 1 | 1 | 1 | 1 | 1 | 1 | 1 | 1 | 1 | 1 |
| MRSA3b | 0 | 0 | 1 | 0 | 0 | 0 | 1 | 0 | 1 | 1 | 0 | 1 | 1 | 1 | 1 | 1 |
| MRSA4b | 1 | 0 | 1 | 0 | 0 | 0 | 1 | 0 | 1 | 1 | 0 | 1 | 1 | 1 | 1 | 1 |
| MRSA5b | 1 | 1 | 1 | 0 | 0 | 0 | 1 | 0 | 1 | 1 | 1 | 0 | 1 | 1 | 1 | 1 |
| MRSA6b | 1 | 1 | 1 | 1 | 1 | 0 | 1 | 1 | 1 | 1 | 1 | 1 | 1 | 1 | 1 | 1 |
| MRSA7b | 0 | 0 | 1 | 0 | 0 | 0 | 1 | 0 | 1 | 1 | 0 | 0 | 1 | 1 | 1 | 1 |
| MRSA8b | 1 | 0 | 1 | 0 | 0 | 0 | 1 | 0 | 1 | 0 | 0 | 0 | 1 | 1 | 1 | 1 |
| MRSA9b | 0 | 0 | 0 | 0 | 0 | 0 | 0 | 0 | 0 | 0 | 0 | 0 | 1 | 0 | 0 | 0 |
| MRSA10b | 1 | 0 | 1 | 0 | 0 | 0 | 1 | 0 | 1 | 1 | 1 | 1 | 1 | 1 | 1 | 1 |
| MRSA11b | 1 | 1 | 1 | 0 | 1 | 0 | 1 | 0 | 1 | 1 | 1 | 1 | 1 | 1 | 1 | 1 |
| MRSA12b | 0 | 0 | 1 | 0 | 0 | 0 | 1 | 0 | 0 | 0 | 0 | 0 | 1 | 1 | 1 | 1 |
| MRSA13b | 1 | 0 | 1 | 0 | 0 | 0 | 1 | 0 | 0 | 0 | 0 | 0 | 1 | 0 | 1 | 0 |
| MRSA14b | 0 | 0 | 1 | 0 | 0 | 0 | 1 | 0 | 0 | 0 | 0 | 0 | 1 | 0 | 1 | 0 |
| MRSA15b | 1 | 1 | 1 | 1 | 1 | 0 | 1 | 1 | 1 | 1 | 1 | 1 | 1 | 1 | 1 | 1 |
| MRSA16b | 1 | 1 | 1 | 0 | 0 | 0 | 1 | 0 | 1 | 1 | 1 | 1 | 1 | 1 | 1 | 1 |
| MRSA17b | 1 | 0 | 1 | 0 | 0 | 0 | 1 | 0 | 0 | 0 | 0 | 0 | 1 | 1 | 1 | 0 |
| MRSA18b | 1 | 1 | 1 | 1 | 0 | 0 | 1 | 0 | 1 | 1 | 1 | 1 | 1 | 1 | 1 | 1 |
| MRSA19b | 1 | 0 | 1 | 0 | 0 | 0 | 1 | 0 | 1 | 1 | 0 | 0 | 1 | 1 | 1 | 1 |
| MRSA20b | 1 | 1 | 1 | 1 | 0 | 0 | 1 | 0 | 1 | 1 | 1 | 1 | 1 | 1 | 1 | 1 |
| MRSA21b | 0 | 0 | 1 | 0 | 0 | 0 | 1 | 0 | 0 | 0 | 0 | 0 | 1 | 0 | 1 | 0 |
| MRSA22b | 1 | 0 | 1 | 0 | 0 | 0 | 1 | 0 | 1 | 1 | 0 | 0 | 1 | 1 | 1 | 1 |
| MRSA23b | 0 | 0 | 1 | 0 | 0 | 0 | 1 | 0 | 0 | 0 | 0 | 0 | 1 | 1 | 1 | 0 |
| MRSA24b | 0 | 0 | 1 | 0 | 0 | 0 | 1 | 0 | 0 | 0 | 0 | 0 | 1 | 1 | 1 | 0 |
| MRSA25b | 0 | 0 | 1 | 0 | 0 | 0 | 1 | 0 | 1 | 1 | 0 | 1 | 1 | 1 | 1 | 1 |
| MRSA26b | 1 | 1 | 1 | 0 | 0 | 0 | 1 | 0 | 1 | 1 | 1 | 1 | 1 | 1 | 1 | 1 |
| MRSA27b | 1 | 1 | 1 | 0 | 0 | 0 | 1 | 0 | 1 | 1 | 0 | 1 | 1 | 1 | 1 | 1 |
| MRSA28b | 1 | 1 | 1 | 1 | 0 | 0 | 1 | 0 | 1 | 1 | 1 | 0 | 1 | 1 | 1 | 1 |
| MRSA29b | 1 | 0 | 1 | 0 | 0 | 0 | 1 | 0 | 1 | 1 | 0 | 0 | 1 | 1 | 1 | 1 |
| MRSA30b | 1 | 1 | 1 | 0 | 0 | 0 | 1 | 0 | 1 | 1 | 0 | 0 | 1 | 1 | 1 | 1 |
| MRSA31b | 0 | 0 | 1 | 0 | 0 | 0 | 1 | 0 | 0 | 0 | 0 | 0 | 0 | 0 | 1 | 0 |
| MRSA32b | 1 | 1 | 1 | 1 | 1 | 0 | 1 | 1 | 1 | 1 | 1 | 1 | 1 | 1 | 1 | 1 |
| MRSA33b | 1 | 1 | 1 | 1 | 1 | 0 | 1 | 1 | 1 | 1 | 1 | 1 | 1 | 1 | 1 | 1 |
| MRSA34b | 1 | 1 | 1 | 1 | 0 | 0 | 1 | 0 | 1 | 1 | 0 | 1 | 1 | 1 | 1 | 1 |
| MRSA35b | 0 | 0 | 1 | 0 | 0 | 0 | 1 | 0 | 0 | 0 | 0 | 0 | 1 | 0 | 1 | 0 |
| MRSA36b | 1 | 1 | 1 | 1 | 0 | 0 | 1 | 0 | 1 | 1 | 1 | 1 | 1 | 1 | 1 | 1 |
| MRSA37b | 0 | 0 | 1 | 0 | 0 | 0 | 1 | 0 | 0 | 0 | 0 | 0 | 1 | 1 | 1 | 0 |
| MRSA38b | 1 | 1 | 1 | 1 | 0 | 0 | 1 | 0 | 1 | 1 | 1 | 1 | 1 | 1 | 1 | 1 |
| MRSA39b | 1 | 1 | 1 | 1 | 1 | 0 | 1 | 1 | 1 | 1 | 1 | 1 | 1 | 1 | 1 | 1 |
| MRSA40b | 1 | 1 | 1 | 0 | 0 | 0 | 1 | 0 | 1 | 1 | 1 | 1 | 1 | 1 | 1 | 1 |
| MRSA41b | 0 | 0 | 1 | 0 | 0 | 0 | 0 | 0 | 0 | 0 | 0 | 0 | 0 | 0 | 0 | 0 |
| MRSA42b | 1 | 1 | 1 | 1 | 1 | 0 | 1 | 1 | 1 | 1 | 1 | 1 | 1 | 1 | 1 | 1 |
| MRSA43b | 1 | 1 | 1 | 0 | 0 | 0 | 1 | 1 | 1 | 1 | 1 | 1 | 1 | 1 | 1 | 1 |
| MRSA44b | 1 | 1 | 1 | 0 | 0 | 0 | 1 | 0 | 1 | 1 | 1 | 1 | 1 | 1 | 1 | 1 |
| MRSA45b | 1 | 1 | 1 | 1 | 0 | 0 | 1 | 0 | 1 | 1 | 1 | 1 | 1 | 1 | 1 | 1 |
| MRSA46b | 1 | 1 | 1 | 0 | 0 | 0 | 1 | 0 | 1 | 1 | 1 | 0 | 1 | 1 | 1 | 1 |
| MRSA47b | 0 | 0 | 1 | 0 | 0 | 0 | 1 | 0 | 0 | 0 | 0 | 0 | 1 | 1 | 1 | 0 |
| MRSA48b | 0 | 0 | 1 | 0 | 0 | 0 | 1 | 0 | 0 | 0 | 0 | 0 | 1 | 1 | 1 | 0 |
| MRSA49b | 0 | 0 | 1 | 0 | 0 | 0 | 1 | 0 | 1 | 1 | 0 | 1 | 1 | 1 | 1 | 1 |

TABLE 12
Validation table of PBP2a MRM transitions from MRSA50b
to MRSA98b strains prepared WITH induction step

| | Transition number | | | | | | | | | | | | | | | |
|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | 16 |
| MRSA50b | 1 | 0 | 1 | 1 | 1 | 1 | 1 | 1 | 1 | 1 | 1 | 1 | 1 | 1 | 1 | 1 |
| MRSA51b | 1 | 1 | 1 | 1 | 1 | 1 | 1 | 1 | 1 | 1 | 1 | 1 | 1 | 1 | 1 | 1 |
| MRSA52b | 0 | 0 | 0 | 0 | 0 | 1 | 1 | 0 | 1 | 0 | 1 | 0 | 1 | 1 | 1 | 1 |
| MRSA53b | 1 | 1 | 1 | 1 | 1 | 1 | 1 | 1 | 1 | 1 | 1 | 1 | 1 | 1 | 1 | 1 |
| MRSA54b | 1 | 1 | 1 | 1 | 1 | 1 | 1 | 1 | 1 | 1 | 1 | 1 | 1 | 1 | 1 | 1 |
| MRSA55b | 1 | 1 | 1 | 1 | 1 | 1 | 1 | 1 | 1 | 1 | 1 | 1 | 1 | 1 | 1 | 1 |
| MRSA56b | 1 | 0 | 0 | 1 | 1 | 1 | 1 | 1 | 1 | 1 | 1 | 1 | 1 | 1 | 1 | 1 |
| MRSA57b | 1 | 1 | 1 | 1 | 1 | 1 | 1 | 1 | 1 | 1 | 1 | 1 | 1 | 1 | 1 | 1 |
| MRSA58b | 1 | 1 | 1 | 1 | 1 | 1 | 1 | 1 | 1 | 1 | 1 | 1 | 1 | 1 | 1 | 1 |
| MRSA59b | 1 | 1 | 1 | 1 | 1 | 1 | 1 | 1 | 1 | 1 | 1 | 1 | 1 | 1 | 1 | 1 |
| MRSA60b | 1 | 1 | 1 | 1 | 1 | 1 | 1 | 1 | 1 | 1 | 1 | 1 | 1 | 1 | 1 | 1 |
| MRSA61b | 1 | 1 | 1 | 1 | 1 | 1 | 1 | 1 | 1 | 1 | 1 | 1 | 1 | 1 | 1 | 1 |
| MRSA62b | 1 | 1 | 1 | 1 | 1 | 1 | 1 | 1 | 1 | 1 | 1 | 1 | 1 | 1 | 1 | 1 |
| MRSA63b | 1 | 1 | 1 | 0 | 0 | 0 | 0 | 0 | 1 | 1 | 1 | 1 | 1 | 1 | 1 | 1 |
| MRSA64b | 1 | 1 | 1 | 0 | 0 | 0 | 0 | 0 | 1 | 1 | 1 | 1 | 1 | 1 | 1 | 1 |
| MRSA65b | 1 | 1 | 1 | 0 | 0 | 0 | 0 | 0 | 1 | 1 | 1 | 1 | 1 | 1 | 1 | 1 |
| MRSA66b | 1 | 1 | 1 | 0 | 0 | 0 | 0 | 0 | 1 | 1 | 1 | 1 | 1 | 1 | 1 | 1 |
| MRSA67b | 1 | 1 | 1 | 0 | 0 | 0 | 0 | 0 | 1 | 1 | 1 | 1 | 1 | 1 | 1 | 1 |
| MRSA68b | 1 | 1 | 1 | 0 | 0 | 0 | 0 | 0 | 1 | 1 | 1 | 1 | 1 | 1 | 1 | 1 |
| MRSA69b | 1 | 1 | 1 | 0 | 0 | 0 | 0 | 0 | 1 | 1 | 1 | 1 | 1 | 1 | 1 | 1 |
| MRSA70b | 1 | 1 | 1 | 0 | 0 | 0 | 0 | 0 | 1 | 1 | 1 | 1 | 1 | 1 | 1 | 1 |
| MRSA71b | 1 | 1 | 1 | 0 | 0 | 0 | 0 | 0 | 1 | 1 | 1 | 1 | 1 | 1 | 1 | 1 |
| MRSA72b | 1 | 1 | 1 | 0 | 0 | 0 | 0 | 0 | 1 | 1 | 1 | 1 | 1 | 1 | 1 | 1 |
| MRSA73b | 1 | 0 | 1 | 0 | 0 | 0 | 0 | 0 | 1 | 1 | 1 | 1 | 1 | 1 | 1 | 1 |
| MRSA74b | 1 | 1 | 1 | 0 | 0 | 0 | 0 | 0 | 1 | 1 | 1 | 1 | 1 | 1 | 1 | 1 |
| MRSA75b | 1 | 1 | 1 | 0 | 0 | 0 | 0 | 0 | 1 | 1 | 1 | 1 | 1 | 1 | 1 | 1 |
| MRSA76b | 1 | 1 | 1 | 0 | 0 | 0 | 0 | 0 | 1 | 1 | 1 | 1 | 1 | 1 | 1 | 1 |
| MRSA77b | 1 | 1 | 1 | 0 | 0 | 0 | 0 | 0 | 1 | 1 | 1 | 1 | 1 | 1 | 1 | 1 |
| MRSA78b | 1 | 1 | 1 | 0 | 0 | 0 | 0 | 0 | 1 | 1 | 1 | 1 | 1 | 1 | 1 | 1 |
| MRSA79b | 1 | 1 | 1 | 0 | 0 | 0 | 0 | 0 | 1 | 1 | 1 | 1 | 1 | 1 | 1 | 1 |
| MRSA80b | 1 | 1 | 1 | 0 | 0 | 0 | 0 | 0 | 1 | 1 | 1 | 1 | 1 | 1 | 1 | 1 |
| MRSA81b | 1 | 0 | 0 | 0 | 0 | 0 | 0 | 0 | 1 | 0 | 1 | 0 | 1 | 1 | 0 | 0 |
| MRSA82b | 1 | 1 | 1 | 0 | 0 | 0 | 0 | 0 | 1 | 1 | 1 | 1 | 1 | 1 | 1 | 1 |
| MRSA83b | 1 | 1 | 1 | 0 | 0 | 0 | 0 | 0 | 1 | 1 | 1 | 1 | 1 | 1 | 1 | 1 |
| MRSA84b | 1 | 1 | 1 | 0 | 0 | 0 | 0 | 0 | 1 | 1 | 1 | 1 | 1 | 1 | 1 | 1 |
| MRSA85b | 1 | 1 | 0 | 0 | 0 | 0 | 0 | 0 | 1 | 1 | 1 | 1 | 1 | 1 | 1 | 1 |
| MRSA86b | 1 | 1 | 1 | 0 | 0 | 0 | 0 | 0 | 1 | 1 | 1 | 1 | 1 | 1 | 1 | 1 |
| MRSA87b | 1 | 1 | 1 | 0 | 0 | 0 | 0 | 0 | 1 | 1 | 1 | 1 | 1 | 1 | 1 | 1 |
| MRSA88b | 1 | 1 | 1 | 0 | 0 | 0 | 0 | 0 | 1 | 1 | 1 | 1 | 1 | 1 | 1 | 1 |
| MRSA89b | 1 | 1 | 1 | 0 | 0 | 0 | 0 | 0 | 1 | 1 | 1 | 1 | 1 | 1 | 1 | 1 |
| MRSA90b | 1 | 1 | 1 | 0 | 0 | 0 | 0 | 0 | 1 | 1 | 1 | 1 | 1 | 1 | 1 | 1 |
| MRSA91b | 1 | 1 | 1 | 0 | 0 | 0 | 0 | 0 | 1 | 1 | 1 | 1 | 1 | 1 | 1 | 1 |
| MRSA92b | 1 | 1 | 1 | 0 | 0 | 0 | 0 | 0 | 1 | 1 | 1 | 1 | 1 | 1 | 1 | 1 |
| MRSA93b | 1 | 1 | 1 | 0 | 0 | 0 | 0 | 0 | 1 | 1 | 1 | 1 | 1 | 1 | 1 | 1 |
| MRSA94b | 1 | 1 | 1 | 0 | 0 | 0 | 0 | 0 | 1 | 1 | 1 | 1 | 1 | 1 | 1 | 1 |
| MRSA95b | 0 | 0 | 0 | 1 | 1 | 1 | 1 | 1 | 1 | 1 | 1 | 1 | 1 | 1 | 1 | 1 |
| MRSA96b | 0 | 0 | 0 | 1 | 1 | 1 | 1 | 1 | 1 | 1 | 1 | 1 | 1 | 1 | 1 | 1 |
| MRSA97b | 0 | 0 | 0 | 1 | 1 | 1 | 1 | 1 | 1 | 1 | 1 | 1 | 1 | 1 | 1 | 1 |
| MRSA98b | 0 | 0 | 0 | 1 | 0 | 1 | 1 | 1 | 1 | 1 | 1 | 1 | 1 | 1 | 1 | 1 |
| | Transition number | | | | | | | | | | | | | | | |
| | 17 | 18 | 19 | 20 | 21 | 22 | 23 | 24 | 25 | 26 | 27 | 28 | 29 | 30 | 31 | 32 |
| MRSA50b | 1 | 1 | 1 | 0 | 0 | 0 | 1 | 0 | 1 | 1 | 0 | 0 | 1 | 1 | 1 | 1 |
| MRSA51b | 1 | 1 | 1 | 0 | 0 | 0 | 1 | 0 | 1 | 1 | 1 | 1 | 1 | 1 | 1 | 1 |
| MRSA52b | 0 | 0 | 1 | 0 | 0 | 0 | 1 | 0 | 0 | 0 | 0 | 0 | 1 | 0 | 1 | 0 |
| MRSA53b | 1 | 1 | 1 | 0 | 0 | 0 | 1 | 0 | 1 | 1 | 0 | 0 | 1 | 1 | 1 | 1 |
| MRSA54b | 1 | 1 | 1 | 1 | 0 | 0 | 1 | 0 | 1 | 1 | 1 | 1 | 1 | 1 | 1 | 1 |
| MRSA55b | 1 | 1 | 1 | 1 | 0 | 0 | 1 | 0 | 1 | 1 | 1 | 1 | 1 | 1 | 1 | 1 |
| MRSA56b | 0 | 0 | 1 | 0 | 0 | 0 | 1 | 0 | 1 | 1 | 0 | 0 | 1 | 1 | 1 | 1 |
| MRSA57b | 1 | 1 | 1 | 1 | 0 | 0 | 1 | 0 | 1 | 1 | 1 | 1 | 1 | 1 | 1 | 1 |
| MRSA58b | 1 | 1 | 1 | 0 | 0 | 0 | 1 | 0 | 1 | 1 | 0 | 1 | 1 | 1 | 1 | 1 |
| MRSA59b | 1 | 1 | 1 | 0 | 0 | 0 | 1 | 1 | 1 | 1 | 1 | 1 | 1 | 1 | 1 | 1 |
| MRSA60b | 1 | 0 | 1 | 0 | 0 | 0 | 1 | 0 | 1 | 1 | 0 | 0 | 1 | 1 | 1 | 1 |
| MRSA61b | 1 | 1 | 1 | 1 | 0 | 0 | 1 | 1 | 1 | 1 | 1 | 1 | 1 | 1 | 1 | 1 |
| MRSA62b | 1 | 1 | 1 | 1 | 0 | 0 | 1 | 1 | 1 | 1 | 1 | 1 | 1 | 1 | 1 | 1 |
| MRSA63b | 1 | 1 | 1 | 0 | 0 | 0 | 1 | 0 | 1 | 1 | 0 | 1 | 1 | 1 | 1 | 1 |
| MRSA64b | 1 | 1 | 1 | 1 | 0 | 0 | 1 | 0 | 1 | 1 | 1 | 1 | 1 | 1 | 1 | 1 |
| MRSA65b | 1 | 1 | 1 | 0 | 0 | 0 | 1 | 0 | 1 | 1 | 1 | 1 | 1 | 1 | 1 | 1 |
| MRSA66b | 1 | 1 | 1 | 1 | 0 | 0 | 1 | 0 | 1 | 1 | 1 | 1 | 1 | 1 | 1 | 1 |
| MRSA67b | 1 | 1 | 1 | 1 | 1 | 0 | 1 | 0 | 1 | 1 | 1 | 1 | 1 | 1 | 1 | 1 |
| MRSA68b | 1 | 1 | 1 | 1 | 0 | 0 | 1 | 1 | 1 | 1 | 1 | 1 | 1 | 1 | 1 | 1 |
| MRSA69b | 1 | 1 | 1 | 0 | 0 | 0 | 1 | 0 | 1 | 1 | 1 | 1 | 1 | 1 | 1 | 1 |
| MRSA70b | 1 | 1 | 1 | 1 | 0 | 0 | 1 | 1 | 1 | 1 | 1 | 1 | 1 | 1 | 1 | 1 |
| MRSA71b | 1 | 1 | 1 | 0 | 0 | 0 | 1 | 0 | 1 | 1 | 1 | 1 | 1 | 1 | 1 | 1 |
| MRSA72b | 1 | 1 | 1 | 1 | 1 | 0 | 1 | 1 | 1 | 1 | 1 | 1 | 1 | 1 | 1 | 1 |
| MRSA73b | 1 | 0 | 1 | 0 | 0 | 0 | 1 | 0 | 0 | 0 | 0 | 0 | 1 | 1 | 1 | 0 |
| MRSA74b | 1 | 1 | 1 | 1 | 0 | 0 | 1 | 0 | 1 | 1 | 1 | 1 | 1 | 1 | 1 | 1 |
| MRSA75b | 1 | 0 | 1 | 0 | 0 | 0 | 1 | 0 | 0 | 0 | 0 | 0 | 1 | 1 | 1 | 1 |
| MRSA76b | 1 | 1 | 1 | 1 | 1 | 0 | 1 | 1 | 1 | 1 | 1 | 1 | 1 | 1 | 1 | 1 |
| MRSA77b | 1 | 1 | 1 | 1 | 0 | 0 | 1 | 0 | 1 | 1 | 1 | 1 | 1 | 1 | 1 | 1 |
| MRSA78b | 1 | 1 | 1 | 1 | 1 | 0 | 1 | 0 | 1 | 1 | 1 | 1 | 1 | 1 | 1 | 1 |
| MRSA79b | 1 | 1 | 1 | 1 | 0 | 0 | 1 | 0 | 1 | 1 | 1 | 1 | 1 | 1 | 1 | 1 |
| MRSA80b | 1 | 0 | 1 | 0 | 0 | 0 | 1 | 0 | 1 | 1 | 0 | 1 | 1 | 1 | 1 | 1 |
| MRSA81b | 0 | 0 | 1 | 0 | 0 | 0 | 1 | 0 | 0 | 0 | 0 | 0 | 1 | 1 | 1 | 0 |
| MRSA82b | 1 | 1 | 1 | 0 | 0 | 0 | 1 | 0 | 1 | 1 | 0 | 1 | 1 | 1 | 1 | 1 |
| MRSA83b | 1 | 0 | 1 | 0 | 0 | 0 | 1 | 0 | 1 | 1 | 0 | 0 | 1 | 1 | 1 | 1 |
| MRSA84b | 1 | 0 | 1 | 0 | 0 | 0 | 1 | 0 | 1 | 1 | 0 | 0 | 1 | 1 | 1 | 1 |
| MRSA85b | 1 | 0 | 1 | 0 | 0 | 0 | 1 | 0 | 0 | 0 | 0 | 0 | 1 | 1 | 1 | 0 |
| MRSA86b | 1 | 1 | 1 | 1 | 0 | 0 | 1 | 0 | 1 | 1 | 1 | 1 | 1 | 1 | 1 | 1 |
| MRSA87b | 1 | 1 | 1 | 1 | 0 | 0 | 1 | 1 | 1 | 1 | 1 | 1 | 1 | 1 | 1 | 1 |
| MRSA88b | 1 | 1 | 1 | 0 | 0 | 0 | 1 | 0 | 1 | 1 | 0 | 0 | 1 | 1 | 1 | 1 |
| MRSA89b | 1 | 1 | 1 | 0 | 0 | 0 | 1 | 0 | 1 | 1 | 0 | 1 | 1 | 1 | 1 | 1 |
| MRSA90b | 1 | 1 | 1 | 0 | 0 | 0 | 1 | 0 | 1 | 1 | 1 | 1 | 1 | 1 | 1 | 1 |
| MRSA91b | 1 | 1 | 1 | 1 | 0 | 0 | 1 | 1 | 1 | 1 | 1 | 1 | 1 | 1 | 1 | 1 |
| MRSA92b | 1 | 1 | 1 | 1 | 1 | 0 | 1 | 1 | 1 | 1 | 1 | 1 | 1 | 1 | 1 | 1 |
| MRSA93b | 1 | 1 | 1 | 1 | 0 | 0 | 1 | 0 | 1 | 1 | 1 | 1 | 1 | 1 | 1 | 1 |
| MRSA94b | 1 | 1 | 1 | 0 | 0 | 0 | 1 | 0 | 1 | 1 | 1 | 1 | 1 | 1 | 1 | 1 |
| MRSA95b | 1 | 1 | 1 | 1 | 0 | 0 | 1 | 1 | 1 | 1 | 1 | 1 | 1 | 1 | 1 | 1 |
| MRSA96b | 1 | 1 | 1 | 0 | 0 | 0 | 1 | 0 | 1 | 1 | 1 | 0 | 1 | 1 | 1 | 1 |
| MRSA97b | 1 | 1 | 1 | 1 | 1 | 0 | 1 | 0 | 1 | 1 | 0 | 1 | 1 | 1 | 1 | 1 |
| MRSA98b | 1 | 0 | 1 | 0 | 0 | 0 | 1 | 0 | 1 | 1 | 0 | 1 | 1 | 1 | 1 | 0 |

Based on the results presented in Tables 9 to 12, the following conclusions can be stated:

Without induction step, 38 MRSA out of 98 exhibit at least 2 positive transitions for at least 3 peptides, which corresponds to a detection sensitivity of 39%.

With the induction step, 96 MRSA out of 98 possess at least 2 positive transitions for at least 3 peptides. This corresponds to a sensitivity of 98% detection of MRSA with these validation criteria. Only the MRSA9b and MRSA41b strains are not correctly identified because only 2 peptides are detected with 2 transitions for the MRSA41b strain and 1 peptide with 2 transitions for the MRSA9b strain.

All the MSSAs analysed using the same method have for each transition, an area under the curve value lower than the threshold values previously set. This means that no MSSA is identified as MRSA, which equates to a specificity of 100%.

### Example 4. MRM3 spectrometry Results for PBP2a

MRM3 transitions for which the area value is greater than the threshold value were considered positive and are denoted as 1. Transitions for which the area is less than or equal to the threshold value are denoted as 0 and considered negative. A sample was considered to be identified as MRSA if at least 1 MRM3 transitions was detected positive. The 19 MSSA strains were analysed according to the same procedure and the results are presented in the following tables.

Table 13 corresponds to the results obtained during the MRM3 analysis of the 98 MRSA without the induction step. It represents the validations of MRM3 transitions of the 98 MRSA prepared WITHOUT the induction step

Table 14 corresponds to the results obtained during the MRM3 analysis of the 98 MRSA with the induction step. It represents the validations of the MRM3 transitions of the 98 MRSA prepared with the induction step.

TABLE 13
MRM3 analysis results without induction step

| | VALELGSK | FQITTSPGSTQK |
|---|---|---|
| | 408, 75/646, | 647, 84/805, |
| | 38/404, 25 | 41/617, 33 |
| MRSA1b | 1 | 1 |
| MRSA2b | 1 | 1 |
| MRSA3b | 1 | 0 |
| MRSA4b | 1 | 1 |
| MRSA5b | 1 | 0 |
| MRSA6b | 1 | 1 |
| MRSA7b | 1 | 0 |
| MRSA8b | 0 | 0 |
| MRSA9b | 0 | 0 |
| MRSA10b | 1 | 1 |
| MRSA11b | 1 | 0 |
| MRSA12b | 1 | 0 |
| MRSA13b | 0 | 0 |
| MRSA14b | 1 | 1 |
| MRSA15b | 1 | 1 |
| MRSA16b | 1 | 1 |
| MRSA17b | 1 | 0 |
| MRSA18b | 1 | 0 |
| MRSA19b | 0 | 0 |
| MRSA20b | 1 | 1 |
| MRSA21b | 1 | 1 |
| MRSA22b | 1 | 0 |
| MRSA23b | 1 | 0 |
| MRSA24b | 0 | 0 |
| MRSA25b | 1 | 1 |
| MRSA26b | 0 | 0 |
| MRSA27b | 1 | 0 |
| MRSA28b | 1 | 1 |
| MRSA29b | 1 | 0 |
| MRSA30b | 1 | 0 |
| MRSA31b | 1 | 1 |
| MRSA32b | 1 | 1 |
| MRSA33b | 1 | 1 |
| MRSA34b | 1 | 1 |
| MRSA35b | 1 | 0 |
| MRSA36b | 1 | 1 |
| MRSA37b | 0 | 0 |
| MRSA38b | 1 | 1 |
| MRSA39b | 1 | 1 |
| MRSA40b | 1 | 1 |
| MRSA41b | 1 | 1 |
| MRSA42b | 1 | 1 |
| MRSA43b | 1 | 1 |
| MRSA44b | 1 | 1 |
| MRSA45b | 1 | 1 |
| MRSA46b | 0 | 0 |
| MRSA47b | 1 | 0 |
| MRSA48b | 1 | 0 |
| MRSA49b | 1 | 0 |
| MRSA50b | 1 | 1 |
| MRSA51b | 1 | 0 |
| MRSA52b | 0 | 0 |
| MRSA53b | 1 | 1 |
| MRSA54b | 1 | 1 |
| MRSA55b | 1 | 0 |
| MRSA56b | 0 | 0 |
| MRSA57b | 1 | 1 |
| MRSA58b | 1 | 1 |
| MRSA59b | 1 | 1 |
| MRSA60b | 1 | 1 |
| MRSA61b | 1 | 1 |
| MRSA62b | 1 | 1 |
| MRSA63b | 1 | 1 |
| MRSA64b | 1 | 1 |
| MRSA65b | 1 | 1 |
| MRSA66b | 1 | 1 |
| MRSA67b | 1 | 1 |
| MRSA68b | 1 | 1 |
| MRSA69b | 1 | 1 |
| MRSA70b | 1 | 0 |
| MRSA71b | 1 | 1 |
| MRSA72b | 1 | 1 |
| MRSA73b | 0 | 0 |
| MRSA74b | 1 | 1 |
| MRSA75b | 1 | 0 |
| MRSA76b | 1 | 1 |
| MRSA77b | 1 | 0 |
| MRSA78b | 1 | 1 |
| MRSA79b | 1 | 1 |
| MRSA80b | 1 | 1 |
| MRSA81b | 1 | 1 |
| MRSA82b | 1 | 1 |
| MRSA83b | 1 | 1 |
| MRSA84b | 1 | 0 |
| MRSA85b | 1 | 1 |
| MRSA86b | 1 | 1 |
| MRSA87b | 1 | 1 |
| MRSA88b | 1 | 0 |
| MRSA89b | 1 | 1 |
| MRSA90b | 1 | 1 |
| MRSA91b | 1 | 1 |
| MRSA92b | 1 | 1 |
| MRSA93b | 1 | 0 |
| MRSA94b | 1 | 1 |
| MRSA95b | 1 | 1 |
| MRSA96b | 1 | 0 |
| MRSA97b | 1 | 0 |
| MRSA98b | 1 | 1 |

TABLE 14
MRM3 analysis results with induction step

| | VALELGSK | FQITTSPGSTQK |
|---|---|---|
| | 408, 75/646, | 647, 84/805, |
| | 38/404, 25 | 41/617, 33 |
| MRSA1b | 1 | 1 |
| MRSA2b | 1 | 1 |
| MRSA3b | 1 | 1 |
| MRSA4b | 1 | 1 |
| MRSA5b | 1 | 1 |
| MRSA6b | 1 | 1 |
| MRSA7b | 1 | 1 |
| MRSA8b | 1 | 1 |
| MRSA9b | 1 | 0 |
| MRSA10b | 1 | 1 |
| MRSA11b | 1 | 1 |
| MRSA12b | 1 | 1 |
| MRSA13b | 1 | 1 |
| MRSA14b | 1 | 1 |
| MRSA15b | 1 | 1 |
| MRSA16b | 1 | 1 |
| MRSA17b | 1 | 1 |
| MRSA18b | 1 | 1 |
| MRSA19b | 1 | 1 |
| MRSA20b | 1 | 1 |
| MRSA21b | 1 | 1 |
| MRSA22b | 1 | 1 |
| MRSA23b | 1 | 1 |
| MRSA24b | 1 | 1 |
| MRSA25b | 1 | 1 |
| MRSA26b | 1 | 1 |
| MRSA27b | 1 | 1 |
| MRSA28b | 1 | 1 |
| MRSA29b | 1 | 1 |
| MRSA30b | 1 | 1 |
| MRSA31b | 1 | 1 |
| MRSA32b | 1 | 1 |
| MRSA33b | 1 | 1 |
| MRSA34b | 1 | 1 |
| MRSA35b | 1 | 0 |
| MRSA36b | 1 | 1 |
| MRSA37b | 1 | 1 |
| MRSA38b | 1 | 1 |
| MRSA39b | 1 | 1 |
| MRSA40b | 1 | 1 |
| MRSA41b | 1 | 1 |
| MRSA42b | 1 | 1 |
| MRSA43b | 1 | 1 |
| MRSA44b | 1 | 1 |
| MRSA45b | 1 | 1 |
| MRSA46b | 1 | 1 |
| MRSA47b | 1 | 1 |
| MRSA48b | 1 | 1 |
| MRSA49b | 1 | 1 |
| MRSA50b | 1 | 1 |
| MRSA51b | 1 | 1 |
| MRSA52b | 1 | 1 |
| MRSA53b | 1 | 1 |
| MRSA54b | 1 | 1 |
| MRSA55b | 1 | 1 |
| MRSA56b | 1 | 1 |
| MRSA57b | 1 | 1 |
| MRSA58b | 1 | 1 |
| MRSA59b | 1 | 1 |
| MRSA60b | 1 | 1 |
| MRSA61b | 1 | 1 |
| MRSA62b | 1 | 1 |
| MRSA63b | 1 | 1 |
| MRSA64b | 1 | 1 |
| MRSA65b | 1 | 1 |
| MRSA66b | 1 | 1 |
| MRSA67b | 1 | 1 |
| MRSA68b | 1 | 1 |
| MRSA69b | 1 | 1 |
| MRSA70b | 1 | 1 |
| MRSA71b | 1 | 1 |
| MRSA72b | 1 | 1 |
| MRSA73b | 1 | 0 |
| MRSA74b | 1 | 1 |
| MRSA75b | 1 | 1 |
| MRSA76b | 1 | 1 |
| MRSA77b | 1 | 1 |
| MRSA78b | 1 | 1 |
| MRSA79b | 1 | 1 |
| MRSA80b | 1 | 1 |
| MRSA81b | 1 | 1 |
| MRSA82b | 1 | 1 |
| MRSA83b | 1 | 1 |
| MRSA84b | 1 | 1 |
| MRSA85b | 1 | 1 |
| MRSA86b | 1 | 1 |
| MRSA87b | 1 | 1 |
| MRSA88b | 1 | 1 |
| MRSA89b | 1 | 1 |
| MRSA90b | 1 | 1 |
| MRSA91b | 1 | 1 |
| MRSA92b | 1 | 1 |
| MRSA93b | 1 | 1 |
| MRSA94b | 1 | 1 |
| MRSA95b | 1 | 1 |
| MRSA96b | 1 | 1 |
| MRSA97b | 1 | 1 |
| MRSA98b | 1 | 1 |

19 methicillin-sensitive strains (MSSA1b, 2b, 3b, 4b, 5b, 6b, 7b, 8b, 9b, 10b, 11b, 12b, 13b, 14b, 15b, 16b, 17b, 18b and 19b) were tested: no peptide derived from PBP2a was detected, as expected. Results that all have a value of zero are not presented in detail.

Without the induction step, 87 strains out of 98 exhibit at least one positive transition, which corresponds to 89% sensitivity (Table 12).

With the induction step, all the MRSA strains tested have at least one positive MRM3 transition, which corresponds to 100% sensitivity (Table 13).

All the MSSA analysed according to the same method (with the induction step) have, for each transition, an area under the curve value less than or equal to the threshold values previously fixed. This means that no sensitive MSSA strain is identified as MRSA, which equates to a specificity of 100%.

### CONCLUSIONS

The performances of the method according to the invention are presented in Table 15 below, which illustrates the importance of the induction step.

TABLE 15
Sensitivity of the method of the invention

Sensitivity of detection of PBP2a

| | | Without induction | With induction |
|---|---|---|---|
| Acquisition | MRM | 39% | 98% |
| method | MRM3 | 89% | 100% |

These results show that the method according to the invention allows a rapid identification of MRSA in less than 1.5 h, directly from a positive blood sample (blood culture), and with performances superior to other methods currently on the market (98% sensitivity and 100% specificity in MRM, and 100% sensitivity and 100% specificity in MRM3).

The implementation of the sample preparation protocol is simple and the cost of consumables per analysis is minimal.

Moreover, the possibility of non-detection in the case of a PBP2a variant protein with one or more point mutations is very unlikely, since the analysis is based on the detection of 8 different peptides of the PBP2a protein.

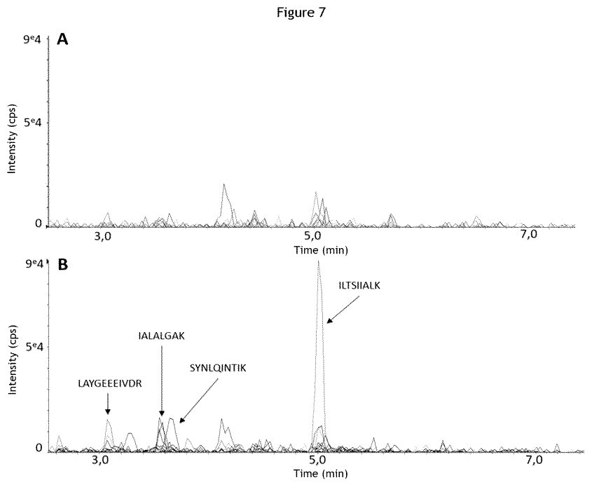

Finally, it has also been shown that the induction step of the method is necessary for the detection of the PBP2c protein in a strain of Staphylococcus aureus expressing the protein. The results presented in FIG. 7 show that detection would have been impossible without the prior step of induction by incubation with an antibiotic of the beta-lactam family.

### REFERENCES

WO 2011/045544
Kirby, W.M.M. (1944). Extraction of a highly potent penicillin inactivator from penicillin resistant staphylococci. Science 99, 452-453.
Felten, A., Grandry, B., Lagrange, P.H., and Casin, I. (2002). Evaluation of Three Techniques for Detection of Low-Level Methicillin-Resistant Staphylococcus aureus (MRSA): a Disk Diffusion Method with Cefoxitin and Moxalactam, the Vitek 2 System, and the MRSA-Screen Latex Agglutination Test. J Clin Microbiol 40, 2766-2771.
Sparbier K, Lange C, Jung J, Wieser A, Schubert S, Kostrzewa M. MALDI biotyper-based rapid resistance detection by stable-isotope labeling. J Clin Microbiol. 2013 Nov;51(11):3741-8.
Tenover, F.C., Jones, R.N., Swenson, J.M., Zimmer, B., McAllister, S., and Jorgensen, J.H. (1999). Methods for Improved Detection of Oxacillin Resistance in Coagulase-Negative Staphylococci: Results of a Multicenter Study. J Clin Microbiol 37, 4051-4058.
Dupieux, C., Bouchiat, C., Larsen, A.R., Pichon, B., Holmes, M., Teale, C., Edwards, G., Hill, R., Decousser, J.-W., Trouillet-Assant, S., et al. (2017). Detection of mecC-Positive Staphylococcus aureus: What To Expect from Immunological Tests Targeting PBP2a? J Clin Microbiol 55, 1961-1963.
Charretier, Y., Dauwalder, O., Franceschi, C., Degout-Charmette, E., Zambardi, G., Cecchini, T., Bardet, C., Lacoux, X., Dufour, P., Veron, L., et al. (2015). Rapid Bacterial Identification, Resistance, Virulence and Type Profiling using Selected Reaction Monitoring Mass Spectrometry. Sci Rep 5, 13944.
Neil JR, Verma A, Kronewitter SR, McGee WM, Mullen C, Viirtola M, Kotovuori A, Friedrich H, Finell J, Rannisto J, Syka JEP, Stephenson JL Jr. Rapid MRSA detection via tandem mass spectrometry of the intact 80 kDa PBP2a resistance protein. Sci Rep. 2021 Sep. 15;11(1):18309.

1. A method for determining the methicillin resistance properties of a strain of Staphylococcus aureus present in a biological sample, comprising the following steps:
a) incubating the biological sample containing said Staphylococcus aureus strain for at least 15 minutes in the presence of an antibiotic from the beta-lactam class chosen from cefoxitin and 6-aminopenicillanic acid (6-APA);
b) isolating the bacteria present in said biological sample;
c) lysing the bacteria and hydrolysing the bacterial proteins in order to obtain a mixture of peptides;
d) analysing this mixture of peptides by targeted mass spectrometry coupled with peptide separation;
wherein the detection of at least one peptide from PBP2a protein having the sequence as shown in SEQ ID NO: 1 or from PBP2c protein having the sequence as shown in SEQ ID NO:2 during the analysis step (d) is indicative of the methicillin resistance of said Staphylococcus aureus strain present in said biological sample.

2. The method according to claim 1, wherein the peptide separation is a peptide separation performed by chromatography or electrophoresis.

3. The method according to claim 1, wherein the mass spectrometry analysis is targeted during the analysis or after the analysis.

4. The method according to claim 1, wherein the incubation step (a) is carried out for a period of 15 minutes to 180 minutes.

5. The method according to claim 1, characterized in that wherein step (b) further comprises a step of selective lysis of non-bacterial cells that are present in the biological sample.

6. The method according to claim 1, wherein the hydrolysis of the bacterial proteins in step (c) is enzymatic hydrolysis.

7. The method according to claim 6, wherein the enzyme is trypsin.

8. The method according to claim 1, wherein the at least one peptide detected is chosen from the group of 12 peptides having the following sequences: SEQ ID NO:3 to SEQ ID NO: 14.

9. The method according to claim 1, wherein the biological sample is chosen from the group consisting of:
blood, serum, plasma, urine, cerebrospinal fluid, tears;
a blood culture, a bacterial colony on agar, a bacterial culture broth;
a food sample; and
any other type of biological sample.

10. A kit for carrying out the method according to claim 1, comprising:
at least one antibiotic of the beta-lactam class chosen from cefoxitin and 6-aminopenicillanic acid (6-APA);
trypsin; and
a reagent allowing selective lysis of non-bacterial cells present in the biological sample.

11. A kit according to claim 10, wherein the reagent allowing selective lysis of non-bacterial cells is a detergent.
